(19) United States
(12) Patent Application Publication
(10) Pub. No.: US 2024/0139310 A1
(43) Pub. Date: May 2, 2024

(54) MICROPARTICLE COMPOSITIONS AND METHODS USE THEREOF

(71) Applicant: Board of Regents of the University of Nebraska, LincoIn, NE (US)

(72) Inventors: Howard E. GENDELMAN, Omaha, NE (US); Bhavesh Kevadiya, Omaha, NE (US); Farah Shahjin, Omaha, NE (US); Milankumar Patel, Omaha, NE (US)

(21) Appl. No.: 18/279,459

(22) PCT Filed: Mar. 9, 2022
(86) PCT No.: PCT/US22/19500

§ 371 Date: Aug. 30, 2023

Related U.S. Application Data
(60) Provisional application No. 63/286,304, filed on Dec. 6, 2021.
(60) Provisional application No. 63/158,484, filed on Mar. 9, 2021.

(51) Int. Cl. A61K 39/215; A61K 9/16; A61P 31/14

(52) U.S. Cl. CPC A61K 39/215; A61K 9/1647; A61K 9/1682; A61P 31/14; A61K 2039/5252

(57) ABSTRACT

The present invention provides porous microparticles, particularly layer-by-layer microparticles, and methods of use thereof.

This application claims priority under 35 U.S.C. § 119(e) to U.S. Provisional Patent Application No. 63/286,304, filed Dec. 6, 2021 and U.S. Provisional Patent Application No. 63/158,484, filed Mar. 9, 2021. The foregoing applications are incorporated by reference herein.

This invention was made with government support under Grants No. R01 MH121402, R01 AG043540, P01 DA028555, R01 NS36126, P01 NS31492, 2R01 NS034239, P01 MH64570, 3P30 MH062261, P30 AI078498, 1R24 OD018546, and R01 AG043540, all awarded by the National Institutes of Health. The government has certain rights in the invention.

### FIELD OF THE INVENTION

The present invention relates generally to the delivery of therapeutics. More specifically, the present invention relates to compositions and methods for the delivery of therapeutic agents to a patient.

### BACKGROUND OF THE INVENTION

Microparticles have been used for the delivery of encapsulated toxic or fragile drugs. However, there is a need to control the release of the encapsulated drug or compound such that there is a longer, sustained release. Improved microparticles with superior release properties are needed.

### SUMMARY OF THE INVENTION

In accordance with the instant invention, porous microparticles comprising one or more polymer layers or sections are provided. In certain embodiments, the microparticle and/or each layer of the microparticle comprises at least one agent or compound. In certain embodiments, the microparticle comprises more than one polymer layer. In certain embodiments, the polymer layer(s) comprises hydrophobic polymers. In certain embodiments, the polymer layer(s) comprises poly(lactide-co-glycolide) (PLGA), poly(L-lactide) (PLLA), or polycaprolactone (PCL). In certain embodiments, the microparticle comprises an external layer of poly(L-lactide) (PLLA)/poly(lactide-co-glycolide) (PLGA) and an internal core of polycaprolactone (PCL). In certain embodiments, the microparticle comprises an external layer of poly(L-lactide) (PLLA)/polycaprolactone (PCL) and an internal core of poly(lactide-co-glycolide) (PLGA). In certain embodiments, the microparticle comprises an external layer of poly(L-lactide) (PLLA) and an internal core of poly(lactide-co-glycolide) (PLGA). In certain embodiments, the microparticle comprises a vaccine such as an inactivated virus. In certain embodiments, the inactivated virus is inactivated SARS-CoV-2. Compositions comprising at least one microparticle and a pharmaceutically acceptable carrier are also encompassed by the instant invention.

According to another aspect of the instant invention, methods of treating, inhibiting, and/or preventing a disease in a subject in need thereof are provided. The methods comprise administering at least one microparticle of the instant invention to the subject.

According to another aspect of the instant invention, methods of synthesizing microparticles are provided. In certain embodiments, the methods comprise a solvent evaporation method.

### BRIEF DESCRIPTIONS OF THE DRAWING

FIGS. 1A-1C shows confirmation of the inactivation of SARS-CoV-2. FIG. 1A: Cytopathic effects of active and inactive virus on Vero E6 cells. Cell death was observed in active virus treated cells from Day 2 (D2) to Day 4 (D4), but there was no change in morphology or viability in the inactive virus treated cells. FIG. 1B: Quantitative PCR for viral RNA expression in Vero E6 cell culture supernatant. For the active viral RNA expression, there is decrease in Ct values depicting increase in the expression. Inactive virus shows no change in expression in 16 hours. FIG. 1C: SDS PAGE of inactive virus for viral spike Si and nucleocapsid protein expression at ˜90 and ˜50 kDa, respectively.

FIG. 2 provides scanning electron micrograph (SEM) of layer-by-layer microparticles (LBL MPs). Top left: External morphology: the MPs (˜50-200 μm) were spherical with pores on its external surface that protrude inwards. Top right: Internal morphology shows the different polymer layers. Porosity also exists in internal layers. Bottom images: Cross section of the LBLs. Outer layer consists of smaller porous balls of polymers. Images are representative of 6 SEM (low and high magnification) images.

FIG. 3 provides SEM images of LBL MPs with the loaded inactive SARS-CoV-2. Top left: External morphology is similar to MPs without the virus. Top right: The internal morphology shows the distinct polymer distribution. Bottom images: High magnification of MPs sections show viral antigen aggregates.

FIG. 4 provides a schematic of the LBL MP. Externally, the MP is spherical and porous. Sectioned MP have the different layers of the polymers, each layer will have the antigen. Biofluid entry through the pores will cause surface erosion and eventual bulk degradation overtime to release the antigen.

FIG. 5A provides Raman microscopy and 3D X-ray microscopy (XRM) images of layer-by-layer microparticles. Left: Brightfield microscopy. Second from left: Fluorescent microscopy. Second from right: Merged image. Right: XRM showing a cross-section of the microparticles. FIG. 5B provides SEM images of in vitro biodegradation of LBL microparticles at different pH conditions between weeks 1-24. The LBL microparticles were subjected to pH 5.5 (potential endosomal pH) and 7.5 (tissue pH) and weekly images were taken to observe the changes in morphology. FIG. 5C provides a graph of an anti-SARS-CoV2 IgG for nucleoprotein in mice injected with the indicated construct. Vehicle: sterile PBS only; LBL: layer-by-layer microparticle without antigen, LBL/Ag: layer-by-layer microparticle with antigen.

FIG. 6 provides SEM images (low and high magnification) of microparticles composed of the polymer PCL produced with the porogen 2-methylpentane (2-MP).

FIG. 7 provides SEM images (low and high magnification) of microparticles composed of the polymer PCL produced with the porogen 3-methylpentane (3-MP).

FIG. 8 provides SEM images (low and high magnification) of microparticles composed of the polymer PCL produced with the porogen 2-methylhexane (2-MH).

FIG. 9 provides SEM images (low and high magnification) of microparticles composed of the polymer PCL produced with the porogen 3-methylhexane (3-MH).

FIG. 10 provides SEM images (low and high magnification) of microparticles composed of the polymer PCL produced with the porogen 2,3-dimethylbutane (2,3-DMB).

FIG. 11 provides SEM images (low and high magnification) of microparticles composed of the polymer PCL produced with the porogen 2,4-dimethylpentane (2,4-DMP).

FIG. 12 provides SEM images (low and high magnification) of microparticles composed of the polymer PLGA produced with the porogen 2-MP.

FIG. 13 provides SEM images (low and high magnification) of microparticles composed of the polymer PLGA produced with the porogen 3-MP.

FIG. 14 provides SEM images (low and high magnification) of microparticles composed of the polymer PLGA produced with the porogen 2-MH.

FIG. 15 provides SEM images (low and high magnification) of microparticles composed of the of polymer PLGA produced with the porogen 3-MH.

FIG. 16 provides SEM images (low and high magnification) of microparticles composed of the polymer PLGA produced with the porogen 2,3-DMB.

FIG. 17 provides SEM images (low and high magnification) of microparticles composed of the polymer PLGA produced with the porogen 2,4-DMP.

FIG. 18 provides SEM images (low and high magnification) of microparticles composed of the polymer PLLA produced with the porogen 2-MP.

FIG. 19 provides SEM images (low and high magnification) of microparticles composed of the polymer PLLA produced with the porogen 3-MP.

FIG. 20 provides SEM images (low and high magnification) of microparticles composed of the polymer PLLA produced with the porogen 2-MH.

FIG. 21 provides SEM images (low and high magnification) of microparticles composed of the polymer PLLA produced with the porogen 3-MH.

FIG. 22 provides SEM images (low and high magnification) of microparticles composed of the polymer PLLA produced with the porogen 2,3-DMB.

FIG. 23 provides SEM images (low and high magnification) of microparticles composed of the polymer PLLA produced with the porogen 2,4-DMP.

### DETAILED DESCRIPTION OF THE INVENTION

The present invention describes novel constructs and formulations for the delivery of compounds such as vaccines, therapeutics, and/or diagnostics. Specifically, the present invention provides polymeric based microparticles, which may optionally be loaded with any components such as a vaccine, therapeutic, and/or diagnostic agent. Microparticles may also be referred to as microspheres.

In accordance with the instant invention, microparticles are provided. In certain embodiments, the microparticles comprises multiple layers (e.g., more than 1) of polymers (e.g., layer-by-layer microparticles). In certain embodiments, the microparticles comprise or encapsulate a compound such as a vaccine, therapeutic, and/or diagnostic agent. In certain embodiments, each layer of the microparticle comprises or encapsulates a compound such as a vaccine, therapeutic, and/or diagnostic agent. In certain embodiments, each layer comprises the same compound such as a vaccine, therapeutic, and/or diagnostic agent.

The multi-layer microparticles of the instant invention allow for the release of the encapsulated compound as the specific layer is degraded. The multilayer microparticles of the instant invention also allow for sustained release of the encapsulated compound. The presence of a multiple layers allows for the delivery of compounds at different times. For example, a compound to be released first can be present within the outer layer. Similarly, a compound to be released last can be present within the inner layer. By varying the distance from the surface of the microparticle, the timing of the release of the compound can be varied. Thus, the multiple layers of the instant invention allow for simultaneous delivery (e.g., when the compounds are in the same coating), delayed delivery (e.g., when the compound is in an inner coating and wherein an outer coating contains an additional compound or only contains polymer), and/or sequential delivery (e.g., when one compound is in an inner coating and a different compound is in an outer coating).

The layers of the microparticles may have different polymer compositions compared to each other and/or comprise different compounds (e.g., a vaccine, therapeutic, and/or diagnostic agent). For example, a microparticle may comprise at least two layers wherein the first and second layers wherein the first and second layers have a different polymer(s) (e.g., the first and second polymer(s) are different) but have the same compounds (e.g., the first and second compound are the same). In certain embodiments, each layer comprises different polymers.

The microparticles of the instant invention may be used for the delivery of the compounds to a cell or subject (e.g., in vitro or in vivo). For example, the microparticles may be administered directly to a subject. In certain embodiments, the microparticle is used for the delivery of a vaccine to a subject. In certain embodiments, the microparticle is administered or delivered to a cell (e.g., autologous cell), which is optionally then delivered to a subject.

Microparticles of the instant invention may have a diameter (e.g., z-average diameter) or a longest dimension of about 1 μm to about 950 μm. In certain embodiments, the microparticles of the instant invention have a diameter of about 1 μm to about 500 μm, about 10 μm to about 500 μm, about 20 μm to about 500 μm, about 25 μm to about 500 μm, or about 30 μm to about 500 μm. In certain embodiments, the microparticles of the instant invention have a diameter of about 1 μm to about 300 μm, about 10 μm to about 300 μm, about 20 μm to about 300 μm, about 25 μm to about 300 μm, or about 30 μm to about 300 μm. In certain embodiments, the microparticles of the instant invention have a diameter of about 50 μm to about 500 μm, about 50 μm to about 450 μm, about 50 μm to about 400 μm, about 50 μm to about 350 μm, about 50 μm to about 300 μm, about 50 μm to about 250 μm, or about 50 μm to about 200 μm.

The microparticles of the instant invention comprise pores. More specifically, the microparticles of the instant invention may comprise pores with controllable pore sizes and pore depths. The pore sizes and pore depths may be controlled based on the type of pore forming agent (porogen) used, the amount of pore forming agent used, and the polymer used in microparticle preparation. Microparticles with controllable pore sizes have wide biomedical and non-biomedical applications. In certain embodiments, each layer of the microparticle comprises pores. Methods of controlling or modulating the pore size and/or frequency (e.g., diameter, depth, and/or density) of microparticles are also encompassed by the instant invention. The methods comprise changing the porogen, polymer, and/or solvent used in the synthesis and/or modulating (e.g., increasing or decreasing) the amount of porogen used (e.g., compared to amount of polymer (e.g., ratio)).

The microparticles of the present invention may contain pores. The pores may also be referred to as wells or holes. The pores may or may not be interconnected. The pores may or may not proceed over the entire surface of the microparticle or throughout each layer. The pores may be in an alignment or arranged throughout the microparticle or on its surface or be present randomly throughout the microparticle or on its surface. In certain embodiments, the pores cover at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95% of the surface of the microparticle, a layer of the microparticle, and/or the entirety of the microparticle. In certain embodiments, the pores cover less than about 95%, less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15%, less than about 10%, less than about 5%, less than about 1%, of the surface of the microparticle, a layer of the microparticle, and/or the entirety of the microparticle.

Pores may be formed in the microparticles using a variety of methods such as the use of organic solvents. Organic solvents used to create pores in microparticles are called porogens. Pores are created in polymeric microparticles when the porogens evaporate from the microparticle leaving behind space where the solvent used to be. Different combinations of porogens and polymers can create different pore sizes. Porogens form pores in a polymer matrix of different pore sizes, depths, and density depending on the polymer type, the amount of porogen used, and the timing of the reaction mixture.

Porous materials may be classified according to their pore sizes: microporous materials have pore sizes of less than 2 nm. Mesoporous materials have pore sizes of between 2 nm and 50 nm. Macroporous materials have pore sizes of between 50 nm and 200 nm. Giga porous materials have pore sizes of greater than 200 nm. The diameter, the number, and the structure of the pores affect the properties of porous microspheres. Pore sizes can affect microparticle absorption, adsorption, permeability, and control drug release or release of an imaging agent, among others. In certain embodiments, the microparticles of the instant invention comprise pore sizes (e.g., diameter or z-average diameter or largest dimension (inclusive of depth)) of about 100 nm to about 5000 nm, about 200 nm to about 5000 nm, about 300 nm to about 4000 nm, or about 400 nm to about 4000 nm.

In certain embodiments, the microparticle porosity can be controlled by using combinations of specific polymers with specific pore forming agents or porogens. In certain embodiments, the porogen is a straight or branched chain alkane or hydrocarbon, particularly wherein the alkane or hydrocarbon comprises 1-10 carbons, 3-10 carbons, 4-10 carbons, 5-10 carbons, or 6-10 carbons. In certain embodiments, the porogen is a branched chain alkane or hydrocarbon, particularly wherein the alkane or hydrocarbon comprises 1-10 carbons, 3-10 carbons, 4-10 carbons, 5-10 carbons, or 6-10 carbons. Examples of porogens include, without limitation, isohexane, 2,4-dimethylpentane, 2,5-dimethylhexane, 2-methylheptane, 2-methylhexane, 2,4-dimethylhexane, 2-methypentane (2-MP), 3-methylpentane (3-MP), 2-methylhexane (2-MH), 3-methylhexane (3-MH), 2,4-dimethylpentane (2,4-DMP), 2,3-dimethylbutane (2,3-DMB), 2-Butanone, and Heptane. In certain embodiments, the porogen is selected from the group consisting of isohexane, 2,4-dimethylpentane, 2,5-dimethylhexane, 2-methylheptane, 2-methylhexane, 2,4-dimethylhexane, 2-methypentane (2-MP), 3-methylpentane (3-MP), 2-methylhexane (2-MH), 3-methylhexane (3-MH), 2,4-dimethylpentane (2,4-DMP), and 2,3-dimethylbutane (2,3-DMB). In certain embodiments, the porogen is selected from the group consisting of isohexane, 2,4-dimethylpentane, 2,5-dimethylhexane, 2-methylheptane, 2-methylhexane, and 2,4-dimethylhexane. In certain embodiments, the porogen is selected from the group consisting of 2-methypentane (2-MP), 3-methylpentane (3-MP), 2-methylhexane (2-MH), 3-methylhexane (3-MH), 2,4-dimethylpentane (2,4-DMP), and 2,3-dimethylbutane (2,3-DMB).

As seen hereinbelow, specific combinations of polymers and porogens result in varying pore sizes. With regard to PCL, PLGA, or PLLA, particles composed of PLGA generally have the shallowest pores, particles composed of PCL generally have larger pores, and particles composed of PLLA generally have the deepest pores but are the most fragile.

The particle size and porosity can be controlled and modified as needed. In certain embodiments, the microparticles are synthesized by a solvent evaporation method (e.g., an organic solvent or a volatile organic solvent). In certain embodiments, the particle size and porosity can be controlled by using specific types of solvents. The solvent type used to manufacture the particles can affect the particle size, wherein the particle size is inversely proportional to the boiling point of the solvent. Solvents that can be used to manufacture the polymers include but are not limited to dimethylformamide (boiling point 153°), ethyl acetate (boiling point 77.5°), tetrahydrofuran (boiling point 66°), chloroform (boiling point 61.2°), to and dichloromethane (boiling point 39.6°). In certain embodiments, the solvent is dichloromethane.

The microparticles of the instant invention may comprise any polymer(s). In certain embodiments, the polymer is biocompatible. In certain embodiments, the polymer is biodegradable. In certain embodiments, the polymer is FDA approved. The polymers of the instant invention may by hydrophobic, hydrophilic, amphiphilic, or mixtures thereof. In certain embodiments, the polymer comprises a hydrophobic polymer. The polymers may be, for example, a homopolymer, random copolymer, blended polymer, copolymer, or a block copolymer. Block copolymers are most simply defined as conjugates of at least two different polymer segments or blocks (e.g., 2 or more repeating units or monomers). The polymer may be, for example, linear, star-like, graft, branched, dendrimer based, or hyper-branched (e.g., at least two points of branching). The polymer of the invention may have, for example, from about 2 to about 10,000, about 2 to about 1000, about 2 to about 500, about 2 to about 250, or about 2 to about 100 repeating units or monomers. The polymers of the instant invention may comprise capping termini.

Examples of hydrophobic polymers include, without limitation: poly(hydroxyethyl methacrylate), poly(N-isopropyl acrylamide), poly(lactic acid) (PLA (or PDLA or PLLA)), poly(lactide-co-glycolide) or poly(lactic-co-glycolic acid) (PLGA), polyglycolide or polyglycolic acid (PGA), polycaprolactone (PCL), poly(aspartic acid), polyoxazolines (e.g., butyl, propyl, pentyl, nonyl, or phenyl poly(2-oxazolines)), polyoxypropylene, poly(glutamic acid), poly(propylene fumarate) (PPF), poly(trimethylene carbonate), polycyanoacrylate, polyurethane, polyorthoesters (POE), polyanhydride, polyester, poly(propylene oxide), poly(caprolactonefumarate), poly(1,2-butylene oxide), poly(n-butylene oxide), poly(ethyleneimine), poly(tetrahydrofurane), ethyl cellulose, polydipyrolle/dicabazole, starch, polyvinylidene fluoride (PVDF), polytetrafluoroethylene (PTFE), polydioxanone (PDO), polyether poly(urethane urea) (PEUU), cellulose acetate, polypropylene (PP), polyethylene terephthalate (PET), nylon (e.g., nylon 6), polycaprolactam, PLA/PCL, poly(3-hydroxybutyrate-co-3-hydroxyvalerate) (PHBV), PCL/calcium carbonate, and/or poly(styrene).

Examples of hydrophilic polymers include, without limitation: polyvinyl alcohol (PVA), polyvinylpyrrolidone (PVP), poly(ethylene glycol) and poly(ethylene oxide) (PEO), chitosan, collagen, chondroitin sulfate, sodium alginate, gelatin, elastin, hyaluronic acid, silk fibroin, sodium alginate/PEO, silk/PEO, silk fibroin/chitosan, hyaluronic acid/gelatin, collagen/chitosan, chondroitin sulfate/collagen, and chitosan/PEO.

Amphiphilic copolymers or polymer composites may comprise a hydrophilic polymer (e.g., segment/block) and a hydrophobic polymer (e.g., segment/block) from those listed above (e.g., gelatin/polyvinyl alcohol (PVA), PCL/collagen, chitosan/PVA, gelatin/elastin/PLGA, PDO/elastin, PHBV/collagen, PLA/hyaluronic acid, PLGA/hyaluronic acid, PCL/hyaluronic acid, PCL/collagen/hyaluronic acid, gelatin/siloxane, PLLA/MWNTs/hyaluronic acid). In a particular embodiment, the amphiphilic block copolymer is an amphiphilic block copolymer comprising hydrophilic poly(ethylene oxide) (PEO) and hydrophobic poly(propylene oxide) (PPO). In a particular embodiment, the polymer is a poloxamer or an amphiphilic triblock copolymer comprising a central hydrophobic PPO block flanked by two hydrophilic PEO blocks (i.e., an A-B-A triblock structure). In a particular embodiment, the amphiphilic block copolymer is selected from the group consisting of Pluronic® L31, L35, F38, L42, L44, L61, L62, L63, L64, P65, F68, L72, P75, F77, L81, P84, P85, F87, F88, L92, F98, L101, P103, P104, P105, F108, L121, L122, L123, F127, 10R5, 10R8, 12R3, 17R1, 17R4, 17R8, 22R4, 25R1, 25R2, 25R4, 25R5, 25R8, 31R1, 31R2, and 31R4.

In certain embodiments, the polymeric solution comprises poly(lactide-co-glycolide) copolymer (PLGA). In certain embodiments, the polymer is PLGA (50:50). The ratio of the lactide and glycolide monomers can be varied. Such variations can tailor the degradation rate of the polymer coating. In a particular embodiment, the ratio of the lactide and glycolide monomers within PLGA is from about 10:90 to about 90:10, particularly about 20:80 to about 80:20, about 30:70 to about 70:30, about 40:60 to about 60:40, about 45:55 to about 55:45, or about 50:50.

In certain embodiments, polymer(s) of the microparticle may include, but is not limited to: poly(lactide-co-glycolide) (PLGA), poly(L-lactide) (PLLA)/polylactic acid (PLA), polycaprolactone (PCL), pluronics/poloxamers, polyesters, poly(propylene oxide), poly(1,2-butylene oxide), poly(n-butylene oxide), poly(tetrahydrofurane), and polystyrene; glyceryl esters, polyoxyethylene fatty alcohol ethers, polyoxyethylene sorbitan fatty acid esters, polyoxyethylene fatty acid esters, sorbitan esters, glycerol monostearate, polyethylene glycols, polypropyleneglycols, cetyl alcohol, cetostearyl alcohol, stearyl alcohol, aryl alkyl polyether alcohols, polyoxyethylene-polyoxypropylene copolymers, poloxamines, cellulose, methylcellulose, hydroxylmethylcellulose, hydroxypropylcellulose, hydroxypropylmethylcellulose, polysaccharides, starch and their derivatives, hydroxyethyl starch, polyvinyl alcohol (PVA), polyvinylpyrrolidone phospholipids, amphiphilic lipids, 1,2-dialkylglycero-3-alkylphophocholines, 1, 2-distearoyl-sn-glecro-3-phosphocholine (DSPC), 1,2-distearoyl-sn-glycero-3-phosphoethanolamine-N-[carboxy(polyethylene glycol) (DSPE-PEG), dimethylaminoethanecarbamoyl cheolesterol (DC-Chol), N-[1-(2,3-Dioleoyloxy)propyl]-N,N,N-trimethylammonium (DOTAP), alkyl pyridinium halides, quaternary ammonium compounds, lauryldimethylbenzylammonium, acyl carnitine hydrochlorides, dimethyldioctadecylammonium (DDAB), n-octylamines, oleylamines, benzalkonium, cetyltrimethylammonium, chitosan, chitosan salts, poly(ethylenimine) (PEI), poly(N-isopropyl acrylamide (PNIPAM), and poly(allylamine) (PAH), poly (dimethyldiallylammonium chloride) (PDDA), alkyl sulfonates, alkyl phosphates, alkyl phosphonates, potassium laurate, triethanolamine stearate, sodium lauryl sulfate, sodium dodecylsulfate, alkyl polyoxyethylene sulfates, alginic acid, alginic acid salts, hyaluronic acid, hyaluronic acid salts, gelatins, dioctyl sodium sulfosuccinate, sodium carboxymethylcellulose, cellulose sulfate, dextran sulfate and carboxymethylcellulose, chondroitin sulfate, heparin, synthetic poly(acrylic acid) (PAA), poly (methacrylic acid) (PMA), poly(vinyl sulfate) (PVS), poly(styrene sulfonate) (PSS), esters and amides of poly(methacrylic acid) and poly(acrylic acid) (e.g., alkylmethacrylates—methyl, ethyl, propyl, butyl) bile acids and their salts, cholic acid, deoxycholic acid, glycocholic acid, taurocholic acid, glycodeoxycholic acid, derivatives thereof, and combinations thereof.

In certain embodiments, the polymers of the different layers of the microparticle have different degradation rates (e.g., in vivo degradation rate). For example, a polymer which is degraded in less than about a month (e.g., less than one week, less than 2 weeks, less than 3 weeks, or less than 4 weeks) may be selected for the external layer of the microparticle. In certain embodiments, a polymer which is degraded in more than a month (e.g., more than 2 months, more than 3 months, more than 4 months, more than 5 months, more than 6 months, more than 9 months, more than 1 year, or more than 2 years) is selected for an internal layer. In certain embodiments, smaller spheres within a layer may be utilized which comprise a polymer which is degraded in more than about 2 years (e.g., PCL).

In certain embodiments, the microparticles comprise PCL. In certain embodiments, the microparticles comprise PLA (e.g., PLLA). In certain embodiments, the microparticles comprise PLGA. In certain embodiments, the microparticles comprise PCL and PLA (e.g., PLLA). In certain embodiments, the microparticles comprise PCL and PLGA. In certain embodiments, the microparticles comprise PLGA and PLA (e.g., PLLA). In certain embodiments, the microparticles comprise PCL, PLGA, and PLA (e.g., PLLA). In certain embodiments, the microparticle comprises a layer of PCL, a layer of PLGA, and a layer of PLA (e.g., PLLA). In certain embodiments, the microparticle comprises an inner core of PLGA, an outer shell of PLA (e.g., PLLA), and PCL spheres incorporated into the outer shell. In certain embodiments, the microparticle comprises an external layer of poly(L-lactide) (PLLA)/poly(lactide-co-glycolide) (PLGA) and an internal core of polycaprolactone (PCL). In certain embodiments, the microparticle comprises an external layer of poly(L-lactide) (PLLA)/25 polycaprolactone (PCL) and an internal core of poly(lactide-co-glycolide) (PLGA).

As stated hereinabove, the microparticles may comprise at least one compound (e.g., a vaccine, therapeutic, and/or diagnostic agent (e.g., detectable agent)). Typically, the compound is contained within the polymeric solution and contained or encapsulated within the polymeric microparticle after synthesis. The compound can be present in the polymeric solution at any concentration. Generally, the polymeric solution will comprise more polymer than compound (w/w). In a particular embodiment, the weight ratio of polymer to agent or compound (e.g., peptide) is from about 1:1 to about 1000:1, about 1:1 to about 100:1, about 1:1 to about 10:1, about 1:1 to about 7.5:1, or about 4:1 or about 5:1 (w/w).

The microparticles of the present invention may be used to package and deliver a compound (e.g., a vaccine, therapeutic, and/or diagnostic agent (e.g., detectable agent)). The compound may be encapsulated, coated, or otherwise packaged in one or more of the polymeric layers of the microparticle. The compound may be, for example, a drug, a nucleic acid molecule, DNA, RNA, a polypeptide, a protein, a small molecule, biologic, growth factor, cytokine, chemokine, viral particle, immunomodulating compound, signaling compound, antibodies, antibody fragments, and/or combinations thereof. In a particular embodiment, the compound is hydrophobic. The various polymer layers can be used to package more than one compound. In certain embodiments, the microparticle encapsulates a vaccine (inactivated virus) or vaccine components. Vaccine and vaccine components include, but are not limited to: inactivated vaccine components, attenuated vaccine components (e.g., live-attenuated virus), proteins, peptides, polysaccharides, capsids, mRNA, DNA, viral vectors, vaccine adjuvants, and/or combinations thereof. In certain embodiments, the vaccine or vaccine components are directed against an infectious organism including but not limited to virus, bacteria, or fungus. In certain embodiments, the vaccine or vaccine component is directed against a virus. Viruses include, but are not limited to: coronaviruses (e.g., SARS-CoV-2, SARS CoV-1, MERS), influenza viruses (e.g., influenza A and influenza B), and Measles virus, Polio virus, Mumps virus, Dengue virus, Ebolavirus, H1N1 virus, Hepatitis A virus, Hepatitis B virus, Hepatitis E virus, Human papillomavirus, Japanese encephalitis virus, Junin virus, Rabies virus, Rotavirus, Rubella virus, encephalitis virus, Varicella zoster virus, Variola virus and Yellow fever virus.

In certain embodiments, the microparticle comprises a SARS-CoV-2 vaccine or vaccine component. In certain embodiments, each layer of the microparticle comprises a SARS-CoV-2 vaccine or vaccine component. In certain embodiments, the microparticle comprises an inactivated SARS-CoV-2 virus (virion). In certain embodiments, each layer of the microparticle comprises an inactivated SARS-CoV-2 virus (virion).

In certain embodiments, the microparticle comprises more than one inactivated virus mutant or variant. In certain embodiments, each layer of the microparticle comprises more than one inactivated virus mutant or variant. In certain embodiments, the microparticle (or each layer of the microparticle) comprises 2, 3, 4, 5, 6, 7, 8, 9, 10, or more inactivated virus mutants or variants. In certain embodiments, the virus is the SARS-CoV-2 virus. Examples of SARS-CoV-2 virus variants include, without limitation: Alpha, Beta, Delta, Gamma, Epsilon, Eta, Iota, Kappa, Zeta, Lambda, Mu, Omicron, and Theta.

The virus (virion) may be inactivated by any method known in the art. For example, the virus (virion) may be inactivated by heat treatment (e.g., pasteurization (e.g., high temperature short time (HTST pasteurization) (e.g., few minutes at 70° C. or higher), low pH treatment (e.g., pH 3.5 or lower for about 1 hour), and/or chemical treatment (e.g., beta-propiolactone). In certain embodiments, the virus is inactivated by beta-propiolactone treatment.

Additional compounds such as therapeutic and/or diagnostic agents can be loaded into the microparticles. Such compounds include, without limitation: small molecules (e.g., BSC-II-IV class drugs for enhancement of bioavailability and sustained/controlled released), antibodies, antibody fragments, antibody-drug conjugates, antigens, proteins, protein subunits, peptides, growth factors, bio/immune modulators, probiotics, therapeutic bacteria, fluorescent dyes, nuclear medicine agents (e.g., PET or SPECT radioisotopes), and combinations thereof. In certain embodiments, a vaccine adjuvant is encapsulated in the microparticle.

Methods of synthesizing the microparticles described herein are also encompassed by the instant invention. Methods of synthesizing microparticles are known in the art. For example, microparticles may be synthesized by microfluidics (e.g., with T-junction devices) and fluid-dynamics methodology. In certain embodiments, the microparticles are synthesized by a solvent evaporation method. In certain embodiments, the method comprises dissolving polymer and porogen in an organic solvent, adding the polymer and porogen mixture to an aqueous solution (e.g., comprising a hydrophilic polymer such as PVA) under stirring until the organic solution evaporates, and obtaining the resultant microparticles (e.g., by centrifugation and/or filtration). The obtained microparticles may be further purified, washed, and/or lyophilized. The methods may further comprise adding a compound to be incorporated into the microparticle. The compound may be added to the polymer and porogen mixture and/or the aqueous solution. In certain embodiments, the compound (e.g., inactivated virus) is added to the aqueous solution. The methods may further comprise the step of inactivating the virus (e.g., as described herein) prior to addition to the microparticle synthesis process.

The instant invention encompasses compositions (e.g., pharmaceutical compositions) comprising at least one microparticle of the instant invention and at least one carrier (e.g., pharmaceutically acceptable carrier). As stated hereinabove, the microparticle may comprise more than one compound. In a particular embodiment, the pharmaceutical composition comprises a first microparticle comprising a first compound and a second microparticle comprising a second compound, wherein the first and second compounds are different (e.g., different inactivated virus mutants or variants). The compositions (e.g., pharmaceutical compositions) of the instant invention may further comprise other compounds or therapeutic agents. For example, the composition may further comprise a vaccine adjuvant.

The present invention also encompasses methods for preventing, inhibiting, and/or treating a disease or disorder. The methods comprise administering a microparticle of the instant invention (optionally in a composition) to a subject in need thereof. In a particular embodiment, the disease or disorder is a viral infection. For example, the microparticles of the instant invention may be administered to a subject to increase the immune response against a viral infection (e.g., SARS-CoV-2). In certain embodiments, the disease or disorder is an infectious disease. In certain embodiments, the infectious disease is caused by a viral infection. Examples of viral infections include, without limitation: coronavirus, influenza virus, measles virus, polio virus, mumps virus, dengue virus, ebolavirus, H1N1 virus, hepatitis a virus, hepatitis b virus, hepatitis e virus, human papillomavirus, Japanese encephalitis virus, junin virus, rabies virus, rotavirus, rubella virus, encephalitis virus, varicella zoster virus, variola virus and yellow fever virus.

The microparticles of the instant invention may also be used, without limitation, in coronary artery stent development, bone tissue regeneration, cartilage growth, and delivery of therapeutics including small molecules, vaccines, and biologics (e.g., cells, DNA, RNA, antibodies, antibody fragments, peptides, proteins). In certain embodiments, microparticles can be loaded with GM-CSF therapeutic protein and administered to a subject to treat neurodegenerative disorders. In certain embodiments, the microparticles can be loaded with fluorescent dyes or tagged molecules for fluorescence activated cell sorting analysis. In certain embodiments, the microparticles can be loaded with cells, particularly stem cells, such as umbilical cord mesenchymal stem cells, and can be used for cell transplantation and/or regenerative medicine by administering the loaded microparticles to a subject in need thereof.

The microparticles of the instant invention (optionally in a composition) can be administered to an animal, in particular a mammal, more particularly a human, in order to treat/inhibit/prevent the disease or disorder. The pharmaceutical compositions of the instant invention may also comprise at least one other compound or therapeutic agent such as an antiviral agent or antimicrobial. The additional antiviral compound may also be administered in a separate pharmaceutical composition from the compositions of the instant invention. The pharmaceutical compositions may be administered at the same time or at different times (e.g., sequentially).

The dosage ranges for the administration of the microparticles and/or compositions of the invention are those large enough to produce the desired effect (e.g., curing, relieving, treating, and/or preventing the disease or disorder, the symptoms of it, or the predisposition towards it). The dosage should not be so large as to cause significant adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counter indications.

The microparticles described herein will generally be administered to a patient as a pharmaceutical composition. The term “patient” as used herein refers to human or animal subjects. These microparticles may be employed therapeutically, under the guidance of a physician.

The pharmaceutical compositions comprising the microparticles of the instant invention may be conveniently formulated for administration with any pharmaceutically acceptable carrier(s). For example, the complexes may be formulated with an acceptable medium such as water, buffered saline, ethanol, polyol (for example, glycerol, propylene glycol, liquid polyethylene glycol and the like), dimethyl sulfoxide (DMSO), oils, detergents, suspending agents, or suitable mixtures thereof, particularly an aqueous solution. The concentration of the microparticles in the chosen medium may be varied and the medium may be chosen based on the desired route of administration of the pharmaceutical composition. Except insofar as any conventional media or agent is incompatible with the microparticles to be administered, its use in the pharmaceutical composition is contemplated.

The dose and dosage regimen of microparticles according to the invention that are suitable for administration to a particular patient may be determined by a physician considering the patient's age, sex, weight, general medical condition, and the specific condition for which the microparticles are being administered and the severity thereof. The physician may also take into account the route of administration, the pharmaceutical carrier, and the microparticle's biological activity.

Selection of a suitable pharmaceutical composition will also depend upon the mode of administration chosen. For example, the microparticles of the invention may be administered by direct injection or to the bloodstream (e.g., intravenously). In this instance, a pharmaceutical composition comprises the microparticle dispersed in a medium that is compatible with the site of injection.

Microparticles of the instant invention may be administered by any method. For example, the microparticles of the instant invention can be administered, without limitation parenterally, subcutaneously, orally, topically, pulmonarily, rectally, vaginally, intravenously, intraperitoneally, intrathecally, intracerebrally, epidurally, intramuscularly, intradermally, or intracarotidly. In a particular embodiment, the microparticle is parenterally. In a particular embodiment, the microparticle is administered orally, intramuscularly, subcutaneously, or to the bloodstream (e.g., intravenously). In a particular embodiment, the microparticle is administered intramuscularly or subcutaneously. Pharmaceutical compositions for injection are known in the art. If injection is selected as a method for administering the microparticle, steps must be taken to ensure that sufficient amounts of the molecules or cells reach their target cells to exert a biological effect. Dosage forms for oral administration include, without limitation, tablets (e.g., coated and uncoated, chewable), gelatin capsules (e.g., soft or hard), lozenges, troches, solutions, emulsions, suspensions, syrups, elixirs, powders/granules (e.g., reconstitutable or dispersible) gums, and effervescent tablets. Dosage forms for parenteral administration include, without limitation, solutions, emulsions, suspensions, dispersions and powders/granules for reconstitution. Dosage forms for topical administration include, without limitation, creams, gels, ointments, salves, patches and transdermal delivery systems.

Pharmaceutical compositions containing a microparticle of the present invention as the active ingredient in intimate admixture with a pharmaceutically acceptable carrier can be prepared according to conventional pharmaceutical compounding techniques. The carrier may take a wide variety of forms depending on the form of pharmaceutical composition desired for administration, e.g., intravenous, oral, direct injection, intracranial, and intravitreal.

A pharmaceutical composition of the invention may be formulated in dosage unit form for ease of administration and uniformity of dosage. Dosage unit form, as used herein, refers to a physically discrete unit of the pharmaceutical composition appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art. In a particular embodiment, the microparticles of the instant invention, due to their long-acting therapeutic effect, need only be administered once to a subject.

Dosage units may be proportionately increased or decreased based on the weight of the patient. Appropriate concentrations for alleviation of a particular pathological condition may be determined by dosage concentration curve calculations, as known in the art.

In accordance with the present invention, the appropriate dosage unit for the administration of microparticles may be determined by evaluating their toxicity in animal models. Various concentrations of microparticles in pharmaceutical composition may be administered to mice, and the minimal and maximal dosages may be determined based on the beneficial results and side effects observed as a result of the treatment. Appropriate dosage unit may also be determined by assessing the efficacy of the microparticle treatment in combination with other standard drugs. The dosage units of nanoparticle may be determined individually or in combination with each treatment according to the effect detected.

The pharmaceutical composition comprising the microparticles may be administered at appropriate intervals until the pathological symptoms are reduced or alleviated, after which the dosage may be reduced to a maintenance level. The appropriate interval in a particular case would normally depend on the condition of the patient.

The instant invention encompasses methods of treating a disease/disorder comprising administering to a subject in need thereof a pharmaceutical composition comprising a microparticle of the instant invention and, particularly, at least one pharmaceutically acceptable carrier. The instant invention also encompasses methods wherein the subject is treated via ex vivo therapy. In particular, the method comprises removing cells from the subject, exposing/contacting the cells in vitro to the microparticles of the instant invention, and returning the cells to the subject. In a particular embodiment, the cells comprise macrophage. Other methods of treating the disease or disorder may be combined with the methods of the instant invention may be co-administered with the pharmaceutical compositions of the instant invention.

The instant invention also encompasses delivering the microparticle of the instant invention to a cell in vitro (e.g., in culture). The microparticle may be delivered to the cell in at least one carrier.

According to another aspect of the instant invention, the porous microspheres of the instant invention are used as micro-scaled scaffolds to support cell attachment and growth (e.g., in vitro). Microspheres allow for cell attachment with higher cell density and cell yields compared with macroscopic scaffolds due to the higher surface area to mass ratio (Huang, et al. (2020) Intl. J. Mol. Sci., 21(5):1895). In certain embodiment, the method comprises seeding or placing cells on the porous microspheres of the instant invention and culturing the cells (e.g., maintaining in a growth media).

The instant invention also encompasses non-biomedical methods and uses of the microparticles presented herein. For example, the microparticles can be used, without limitation, in petrochemical industries for gas adsorption, waste disposal, catalysts, energy conversion, in gas storage devices, molecular sieves and absorption of water pollutants. In certain embodiments, the microparticles of the instant invention can be used for CO₂ and/or methane gas absorption in petrochemical and/or food waste industries.

### Definitions

The following definitions are provided to facilitate an understanding of the present invention.

The singular forms “a,” “an,” and “the” include plural referents unless the context clearly dictates otherwise.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., polysorbate 80), emulsifier, buffer (e.g., Tris HCl, acetate, phosphate), antimicrobial, bulking substance (e.g., lactose, mannitol), excipient, auxiliary agent or vehicle with which an active agent of the present invention is administered. Pharmaceutically acceptable carriers can be sterile liquids, such as water and oils, including those of petroleum, animal, vegetable or synthetic origin. Water or aqueous saline solutions and aqueous dextrose and glycerol solutions are preferably employed as carriers, particularly for injectable solutions. Suitable pharmaceutical carriers are described in “Remington's Pharmaceutical Sciences” by E. W. Martin (Mack Publishing Co., Easton, PA); Gennaro, A. R., Remington: The Science and Practice of Pharmacy, (Lippincott, Williams and Wilkins); Liberman, et al., Eds., Pharmaceutical Dosage Forms, Marcel Decker, New York, N.Y.; and Kibbe, et al., Eds., Handbook of Pharmaceutical Excipients, American Pharmaceutical Association, Washington.

The term “treat” as used herein refers to any type of treatment that imparts a benefit to a patient afflicted with a disease, including improvement in the condition of the patient (e.g., in one or more symptoms), delay in the progression of the condition, etc. In a particular embodiment, the treatment of a viral infection results in at least an inhibition/reduction in the number of infected cells and/or detectable viral levels.

As used herein, the term “prevent” refers to the prophylactic treatment of a subject who is at risk of developing a condition (e.g., viral infection) resulting in a decrease in the probability that the subject will develop the condition.

A “therapeutically effective amount” of a compound or a pharmaceutical composition refers to an amount effective to prevent, inhibit, treat, or lessen the symptoms of a particular disorder or disease. The treatment of a viral infection herein may refer to curing, relieving, and/or preventing the viral infection, the symptom(s) of it, or the predisposition towards it.

As used herein, the term “therapeutic agent” refers to a chemical compound or biological molecule including, without limitation, nucleic acids, peptides, proteins, and antibodies that can be used to treat a condition, disease, or disorder or reduce the symptoms of the condition, disease, or disorder.

As used herein, the term “small molecule” refers to a substance or compound that has a relatively low molecular weight (e.g., less than 4,000, less than 2,000, particularly less than 1 kDa or 800 Da). Typically, small molecules are organic, but are not proteins, polypeptides, or nucleic acids, though they may be amino acids or dipeptides.

The term “antimicrobials” as used herein indicates a substance that kills or inhibits the growth of microorganisms such as bacteria, fungi, viruses, or protozoans.

As used herein, the term “antiviral” refers to a substance that destroys a virus and/or suppresses replication (reproduction) of the virus. For example, an antiviral may inhibit and or prevent: production of viral particles, maturation of viral particles, viral attachment, viral uptake into cells, viral assembly, viral release/budding, viral integration, etc.

As used herein, the term “amphiphilic” means the ability to dissolve in both water and lipids/apolar environments. Typically, an amphiphilic compound comprises a hydrophilic portion and a hydrophobic portion. “Hydrophobic” designates a preference for apolar environments (e.g., a hydrophobic substance or moiety is more readily dissolved in or wetted by non-polar solvents, such as hydrocarbons, than by water). “Hydrophobic” compounds are, for the most part, insoluble in water. As used herein, the term “hydrophilic” means the ability to dissolve in water.

As used herein, the term “polymer” denotes molecules formed from the chemical union of two or more repeating units or monomers. The term “block copolymer” most simply refers to conjugates of at least two different polymer segments, wherein each polymer segment comprises two or more adjacent units of the same kind.

An “antibody” or “antibody molecule” is any immunoglobulin, including antibodies and fragments thereof (e.g., scFv), that binds to a specific antigen. As used herein, antibody or antibody molecule contemplates intact immunoglobulin molecules, immunologically active portions of an immunoglobulin molecule, and fusions of immunologically active portions of an immunoglobulin molecule.

As used herein, the term “immunologically specific” refers to proteins/polypeptides, particularly antibodies, that bind to one or more epitopes of a protein or compound of interest, but which do not substantially recognize and bind other molecules in a sample containing a mixed population of antigenic biological molecules.

The following examples provide illustrative methods of practicing the instant invention and are not intended to limit the scope of the invention in any way.

### Example 1

Severe acute respiratory syndrome coronavirus 2 (SARS-CoV-2) is the etiological agent for COVID-19 pandemic with devastating consequences globally (Zhu, et al. (2020) Glob. Health Res. Policy, 5:6). While most cases of SARS-CoV-2 infections are either asymptomatic or lead to mild signs and symptoms, those in their seventh or beyond decades of life and with known co-morbidities such as hypertension, diabetes mellitus, chronic lung disease, cancer or with concurrent immune suppression, show higher frequencies of life-threatening end organ disease (Mueller, et al. (2020) Aging 12:9959-9981). This includes, most commonly, the acute respiratory distress syndrome (ARDS), causing pulmonary demise and untimely death (Kevadiya, et al. (2021) J. Neuroimmune Pharmacol., 16:12-37). This has claimed the lives of over two million individuals worldwide.

Available effective treatments are limited. Control measures such as the use of masks, physical distancing, testing of exposed or symptomatic persons, contact tracing, and isolation have helped limit the transmission. However, there is still no antiviral agent capable of ensuring the proper treatment or full protection against virus. SARS-COV-2 are continually under genetic mutations and produced new variants with different characteristics that may lead to spread faster and cause more severe disease or may escape from host immune response (Dos Santos, W. G. (2021) Biomed. Pharmacother., 136:111272-111272). To mitigate the effects of the virus on public health, the race is to develop a vaccine capable of effective herd immunity.

Currently, there is still no antiviral agent capable of ensuring the proper treatment or full protection against SARS-CoV-2. The COVID-19 pandemic has required rapid action and the vaccines development in an unprecedented timeframe (Krammer, F. (2020) Nature 586:516-527). Three SARS-CoV-2 vaccines are immediately available while others are in varying phases of preclinical or clinical trial development (Baden, et al. (2020) New Engl. J. Med., 384:403-416; Polack, et al. (2020) New Engl. J. Med., 383:2603-2615; Ramasamy, et al. (2020) Lancet 396:1979-1993; Chakraborty, et al. (2021) Adv. Drug Del. Rev., 172:314-338; Gao, et al. (2020) Science 369:77-81; Kyriakidis, et al. (2021) Vaccines 6:28). This includes nucleic acid-, lipid nanoparticle-, protein-subunit-, and viral vector-based vaccines that protect against viral infection (Dong, et al. (2020) Signal Transd. Targeted Ther., 5:237; Rubin, et al. (2020) New Engl. J. Med., 383:2677-2678). Pfizer and Moderna pioneered mRNA vaccines that are safe and effective against SARS-CoV-2 infections (Polack, et al. (2020) New Engl. J. Med., 383:2603-2615). Despite the recorded efficacy, there remain a number of limitations (He, et al. (2006) J. Virology 80:5757; Ravichandran, et al. (2020) Sci. Trans. Med., 12:eabc3539; Dai, et al. (2021) Nat. Rev. Immun., 21:73-82; Logunov, et al. (2021) Lancet 397:671-681). First, the vaccines were given emergency use and their duration of action remains uncertain. Second, each of the deployed vaccines focuses on a single spike protein as the induction immunogen and requires repeated doses. Third, there is a noted lack of long-term memory cell responses that are known to prevent reinfection. Such responses remain difficult to assess as cross-reactive CD4+ memory T cells and herd immunity both impact transmission. Fourth, there is no long-term toxicity profiles. Fifth, known SARS-CoV-2 genomic variabilities and linked host immune response show a real potential for limitations in long-term efficacy. Moreover, there are no late-stage vaccine trials to avail the vaccine safety and efficacy data for any of the vaccines (Polack, et al. (2020) New England J. Med., 383:2603-2615; Forni, et al. (2021) Cell Death & Differentiation 28:626-639). Thus, there is an immediate need to develop alternative safe and effective vaccines that consider viral genomic variability, toxicities and induction of antiviral CD4+ T cell memory cells.

Herein, a vaccine strategy is provided that addresses and overcomes all of the above drawbacks. The strategy allows (i) broad epitope coverage to elicit immune response, (ii) pulsatile/sustained release of antigen to elicit sustained immune response, and (iii) retain flexibility for inclusion of mutant variants, if any appears to spread within the population. A vaccine approach is provided that deploys multiple distinct viral antigens as immunogens, which is accomplished by slow-release layer-by-layer microparticles (LBL MPs). This approach allows for the delivery of “whole” inactivated SARS-CoV-2 for broad antigen exposures. Inactivated viruses-based vaccines are traditionally safe and effective for protecting against influenza, poliovirus, and other viruses (Sanders, et al., Inactivated Viral Vaccines. Vaccine Analysis: Strategies, Principles, and Control (2014) 45-80). However, until now, there is lack of studies investigating the biodegradable multilayered polymer-based MPs designed for pulsatile antigen release as vaccine candidate (Khademi, et al. (2018) Iran J Basic Med Sci., 21:116-123; Han, et al. (2018) Polymers (Basel) 10:31; Gao, et al. (2018) Frontiers Immun., 9:345; Malyala, et al. in Immunopotentiators in Modern Vaccines (Second Edition) (eds Schijns et al.) 231-248, Academic Press (2017); Zhang, et al. (2021) Lancet Infect Dis., 21:181-192). The present strategy allows for broad immune responses, pulsatile release of viral antigens, and a sustained memory and humoral antiviral immune response for insertion of predicted viral variants. Therefore, the approach employs pulsatile release of SARS-CoV-2 antigens from polymeric layer MPs with sustained degradation capable of eliciting a sustained antiviral immune response.

The SARS-CoV-2 enters through the naso-oral route, followed by the infection of the ACE2 receptor expressing type 2 alveolar cells in lungs (Shah, et al. Frontiers Immun., 11:1949). These viruses propagate and are engulfed by the antigen presenting cells (APCs) when released from the host cells. The APCs initiate a cascade of humoral and cell mediated immune response (Lees, et al. (2010) Immunology 130:463-470). The APCs present the viral antigen to the naïve T cells. These T cells release cytokines to (i) activate B cell response (release the virus specific antibodies to neutralize the infectious agent) and (ii) activate CD8+ T cell response (clearance of the infected cells). Both these responses also result in production of memory B- and T-cells that patrol the body to prevent reinfection. Therefore, the pulsatile release of the antigen (whole, inactivated virus) from the individual polymeric layers (with different degradation rates) will elicit a sustained immune response to SARS-CoV-2.

SARS-COV-2 is undergoing constant mutations and new strains are generated every few months in different places around the world. It would be difficult to manufacture mRNA-based vaccine for each new strain that develops. Moreover, most vaccines are intended to provide protection against reinfection for long periods. However, currently available vaccines require multiple dosing and there are no long-term data to avail the status of the immune system to provide the much-needed long term protection. Therefore, there is the need for a single-dose, long-term protective, effective vaccine platform that can easily accommodate any strain of the virus. The delivery vehicle provided herein that is multilayered, allowing pulsatile release of the antigen of interest for prolong periods of time. Multilayer designs have been tried for the delivery of drugs with sustained release in vivo (Park, et al. (2018) Biomaterials Res., 22:29). However, ‘whole inactivated’ virus has not been delivered by such constructs. Here, inactive SARS-CoV-2 loaded LBL MPs are provided. These LBL MPs will induce virus-specific neutralizing antibodies. Further, the LBL MPs will allow for the inclusion of the multiple mutant variants of SARS-CoV-2, which have complicated the effectiveness of the existing vaccines. The present approach can be translational, allowing the clinical testing of MPs-based single dose vaccines.

### Inactivation of SARS-CoV-2

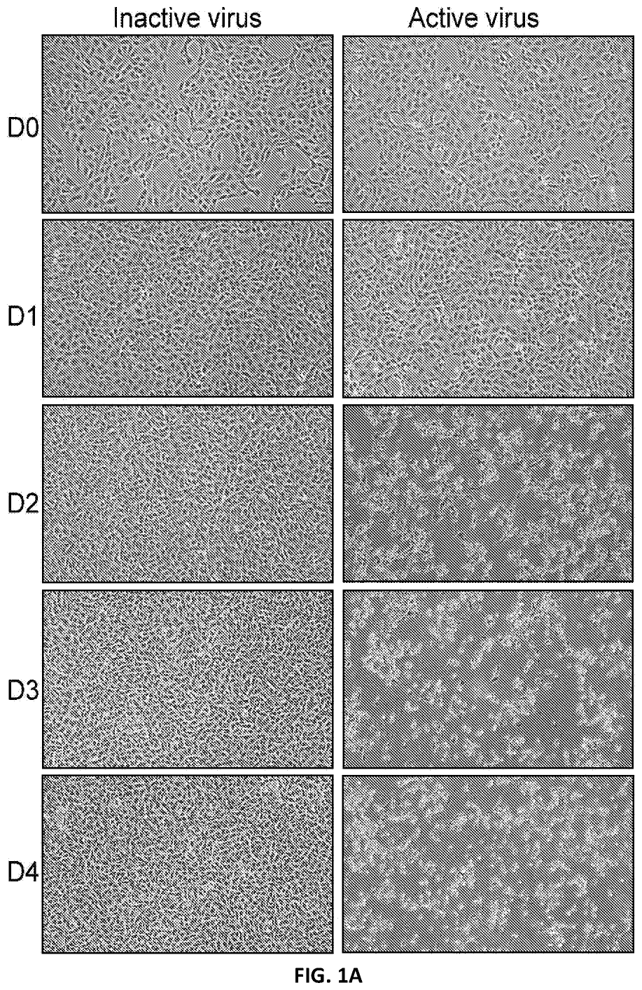

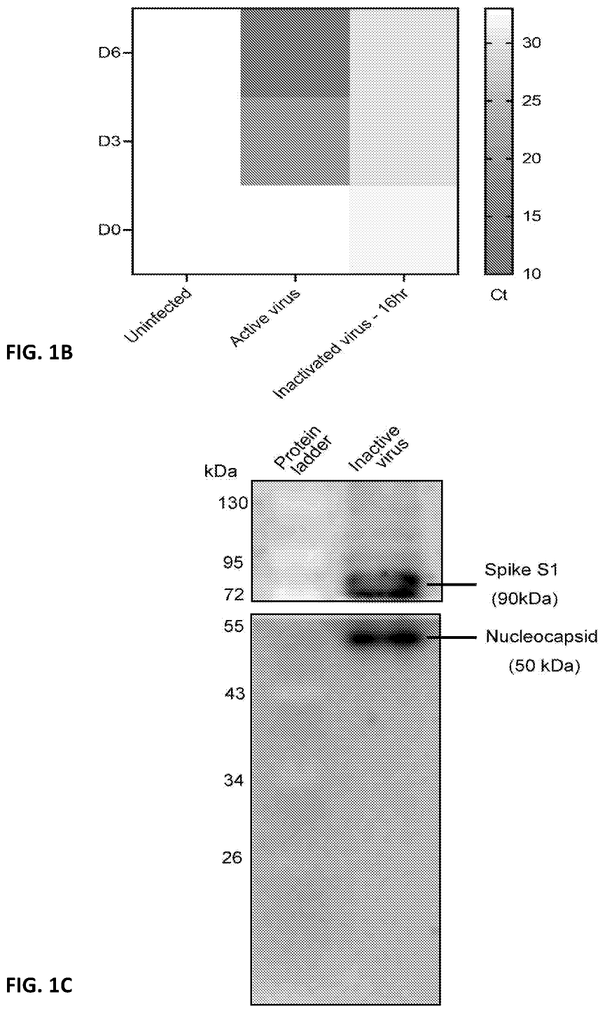

SARS-CoV-2 (strain 2019-nCoV/USA-WA1/2020) was inactivated using β-propiolactone (BPL), which retains the viral antigenicity (Jureka, et al. (2020) Viruses 12:622). Specifically, incubation of SARS-CoV-2 (1×10⁶ pfu) in solution with 0.5% BPL for 16 hours at 4° C. was followed by a 2 hour incubation at 37° C. This resulted in complete inactivation of infectious SARS-CoV-2 (Jureka, et al. (2020) Viruses 12:622; Gao, et al. (2020) Science 369:77-81). Inactivated virus was purified by high-speed centrifugation over 20% sucrose gradient. The inactivation was confirmed by several assays before taking the virus out of the BSL-3 facility. Cytopathic effects of the active and inactive virus (at multiplicity of infection (MOI)=0.01) over the period of 5 days was observed in Vero E6 cells (Ogando, et al. (2020) J. Gen. Virol., 101:925-940). As seen in FIG. 1A, cell apoptosis, detachment and death were observed for the active virus treated Vero E6 cells. In contrast, for the inactive virus treated cells, there were no changes in cell morphology or viability (FIG. 1A).

Next, the cells were treated with active or inactive virus (at MOI=0.01) and supernatants were collected at days 0, 3 and 6. Real time qPCR was performed to quantify viral RNA in supernatants of the Vero E6 cell culture over the period of 6 days. It was observed that for the active virus, there was increased viral RNA expression (decrease in Ct values), meaning viral propagation was occurring. For the inactive virus, there was no change in the viral RNA expression at days 0, 3 or 6 (FIG. 1B), indicating no propagation. A plaque assay was also performed to confirm inactivation. To demonstrate that the antigenic epitopes are not destroyed by the inactivation process, it was shown that both nucleoprotein (N) and spike (S) proteins of inactive SARS-CoV-2 are intact and detectable by western blot post-inactivation (FIG. 1C).

### Preparation and Characterization of SARS-CoV-2 Virus Encased Layer-by-Layer Microparticles

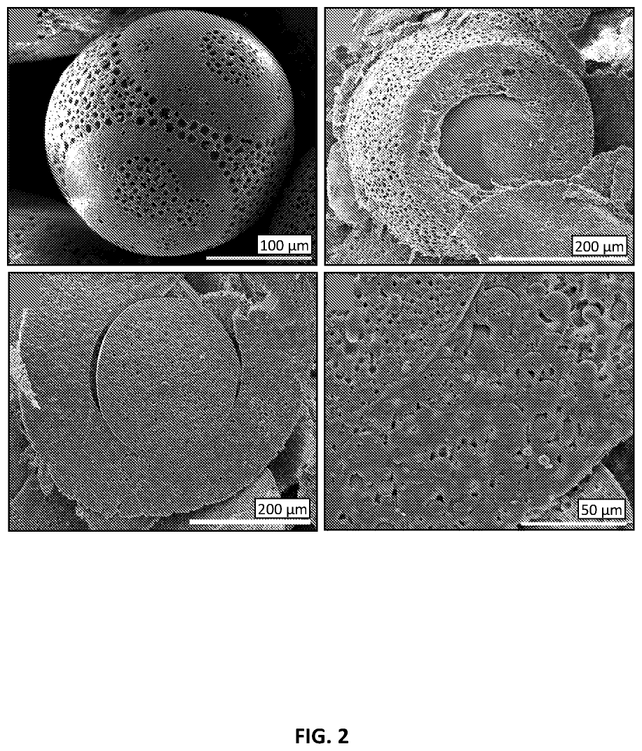

For the preparation of the particles, the polymers selected are biodegradable, biocompatible and FDA approved for use in drug and vaccine delivery and surgical devices (Middleton, et al. (2000) Biomaterials 21:2335-2346; Marin, et al. (2013) Int J Nanomedicine 8:3071-3090). Each polymer layer of the particle undergoes biodegradation at different time points. Polycaprolactone (PCL), polylactide (PLLA), and poly(lactic-co-glycolic acid) (PLGA) were made into LBL MPs by a standard solvent evaporation method carried out in polyvinyl alcohol solution (PVA). The porous morphology was achieved using isohexane during the preparation. The particle pellets obtained after centrifugation were characterized using scanning electron microscopy (SEM) (FIG. 2). The SEM reflects the external PCL layer with PLLA incorporations as smaller balls within PCL layers (Yoshioka, et al. (2010) Polymer Engr. Sci., 50:1895-1903; Lee, et al. (2010) Acta Biomaterialia 6:1342-1352).

More specifically, the LBL MPs were prepared as follows. First, PCL (100 mg), PLGA (100 mg), and PLLA (50 mg) were dissolved in 1 ml DCM. 50 μL to 500 μL isohexane or 2,4-dimethylpentane or 2,5-dimethylhexane or 2-methylheptane or 2-methylhexane or 2,4-dimethylhexane was mixed thoroughly to the DCM-polymer mix. Second, the resulting polymer mixture was poured into 1% w/v polyvinyl alcohol (300 ml) aqueous solution while being stirred at 1000 rpm. Third, the mechanical stirring (at max speed 1300 rpm) at 25.0° C. was continued for 6-16 hours to completely evaporate the organic solvent and to obtain the solid microparticles. Fourth, the solid microparticles were centrifuged at 2000 rpm (alternatively filtered using Whatman paper) and the pellet was lyophilized and subjected to characterization.

Alternatively, to produce smaller particles with internal PCL and external PLGA/PLLA layered microparticles, additional 2 mL dimethylformamide (DMF) was added to the organic mixture. As such, it possible to have the desired arrangement of layers from internal to external.

### Incorporation of SARS-CoV-2 in the LBL MPs

LBL MPs are vehicles for peptide-based therapeutics (Panyam, et al. (2003) J Control Release 92:173-187; Primavera, et al. (2018) Diabetes 67:2279; Raghuvanshi, et al (2002) Int J Pharm., 245:109-121). Inactive virus was incorporated into the LBL MPs using the same solvent evaporation method as described above. Specifically, 20% w/w of antigen (inactive virus) was added to polymer mass for the preparation.

More specifically, LBL-virus preparations were prepared as follows.
1. SARS-CoV-2 was cultured in Vero E6 cells. Supernatant of the infected cell culture was collected at day 5 post infection. The supernatant was centrifuged at 2000 rpm to remove cell debris. Plaque assays were performed to determine the plaque forming units (PFU). Based on the PFU counts, 0.5% v/v beta-propiolactone was added per 1×10⁶ PFU to inactivate the virus in supernatant. The active virus was incubated with beta-propiolactone for 16 hours and then 2 hours in 37° C. incubator to inactivate the beta-propiolactone. Inactivation confirmation assays—plaque assay, cytopathic assay, qPCR—were performed. The supernatant (containing inactive virus) was filtered through 0.45 μm sterile polyvinylidene difluoride (PVDF) membrane filter to remove large organelles and remnant cell debris. Finally, high speed centrifugation at 100,000×g in 20% sucrose gradient was performed to obtain pure viral particles. The viral particles were resuspended in Tris-NaCl-EDTA buffer and quantified using NanoDrop™.
2. PCL (100 mg), PLGA (100 mg), and PLLA (50 mg) were dissolved in 1 ml dichloromethane (DCM). 50 μL to 500 μL isohexane or 2,4-dimethylpentane or 2,5-dimethylhexane or 2-methylheptane or 2-methylhexane or 2,4-dimethylhexane was mixed thoroughly to the DCM-polymer mix.
3. Inactive virus (equivalent to 20-300 mg viral proteins) was added to 1% w/v polyvinyl alcohol (300-1000 ml) aqueous solution.
4. The resulting polymer mixture was poured into 1% w/v polyvinyl alcohol (300 ml) aqueous solution (containing inactive virus) while being stirred at 1000 rpm.
5. The mechanical stirring (at max speed 1300 rpm) at 25.0° C. was continued for 6-16 hours to completely evaporate the organic solvent and to obtain the solid microparticles.
6. The solid microparticles were centrifuged at 2000 rpm (alternatively filtered using Whatman paper). The pellet was lyophilized and subjected to characterization.

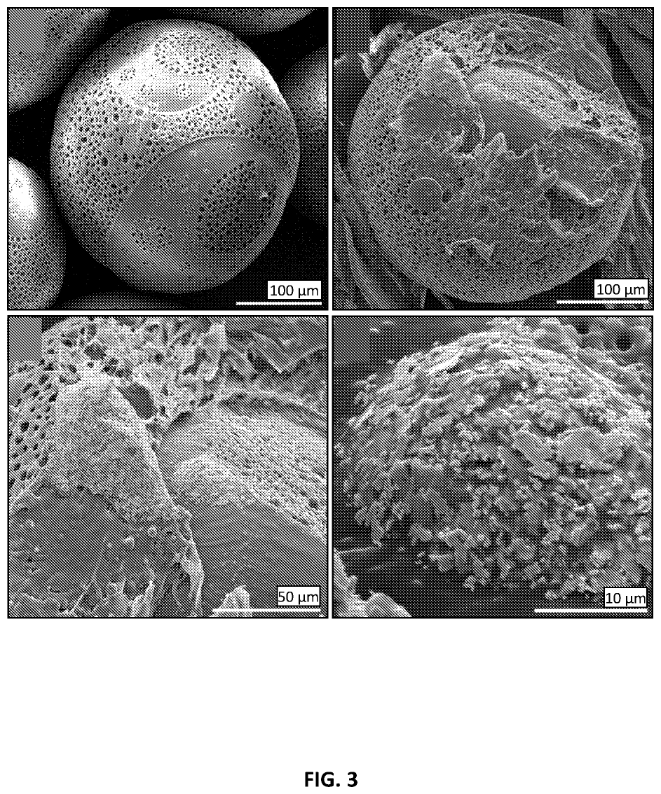

The MPs were characterized by SEM. Antigen aggregates were observed on polymer surfaces between layers within the MPs (FIG. 3).

LBL MPs are finding widespread importance in the delivery of encapsulated toxic or fragile drugs (Becker, et al. (2009) Langmuir 25:14079-14085; Chua, et al. (2015) Biomaterials 53:50-57). The effectiveness of LBL MPs as therapeutic delivery vehicles is often dependent on the degradation behavior of the layers because it is often necessary to acquire temporal and spatial control in release of the encapsulated therapeutic agents. The polymers used herein have different rates of degradation, ranging from a few weeks to several months (Becker, et al. (2009) Langmuir 25:14079-14085; Chua, et al. (2015) Biomaterials 53:50-57).

Without being bound by theory, the polyester-polymers' degradation occurs by the following steps. At first, the polymer hydration causes the disruption of the structure by disrupting hydrogen bonds and van der Waals forces. There is also a loss of mechanical strength caused by the rupture of covalent bonds of the polymer backbone brought about by hydrolysis in vivo. Subsequently, there is polymer mass loss resulting in accelerated water absorption, polymer dissolution and/or phagocytosis (DeLuca, et al. (1993) in Polymeric Delivery Systems Vol. 520 ACS Symposium Series, Ch. 4, 53-79 (American Chemical Society); D'Souza, et al. (2015) Advances in Pharmaceutics 2015:154239). Eventually, hydrolytic degradation of these biocompatible polyesters produces lactic and glycolic acids, both moieties cleared in vivo by the Krebs cycle (Netti, et al. (2020) Polymers (Basel) 12:2042). With MPs administration, the mechanism of dispersion may occur by either (i) release of immunogen from MPs at injection site, or (ii) phagocytosis of MPs by APCs, with subsequent migration away from the injection site (Wang, et al. (2014) Biomaterials 35:8385-8393).

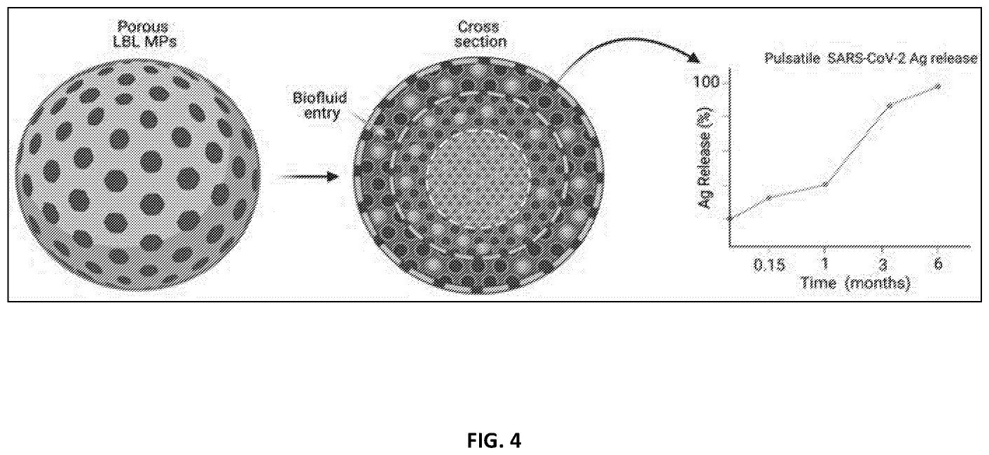

Considering the particles have different degradation rates, the layers will undergo surface erosion, followed by bulk degradation. The external layer will be degraded first and release inactive virus (e.g., in less than a month), followed by the internal layer degradation at a later time point (e.g., 6-8 months later) to release more of the immunogen. Raman mapping can be used to confirm that the internal layer is PLGA (most dense among the three polymers chosen), external layer is PLLA and the balls within PCL to be PLLA (FIG. 4). PCL degradation is the slowest and those particles may be released and dispersed from the site of injection, independently or via APCs, and can release the immunogen away from the site of injection. The use of LBL MPs to deliver ‘whole’ inactive virus is unprecedented.

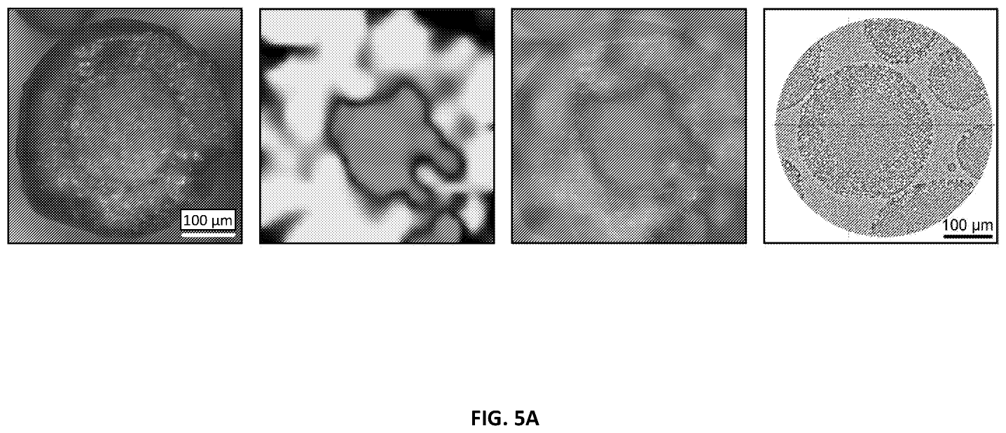

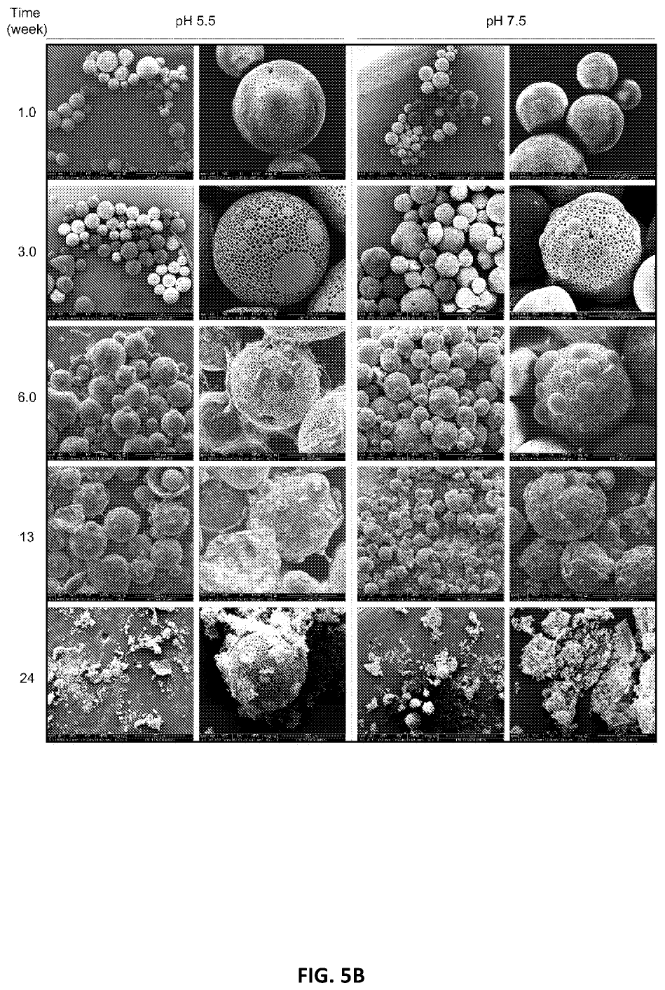

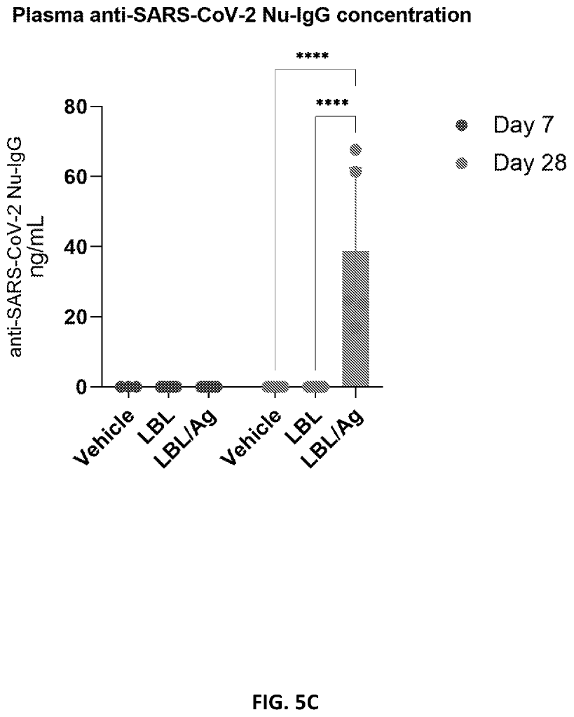

To further characterize the LBL MPs, Raman microscopy and 3D X-ray diffraction microscopy (XRM) were performed (FIG. 5A). Brightfield (left) and fluorescent microscopy (second from left) both show distinct polymer layers (external=PCL; internal=PLGA). Merged image is second from right. The layered morphology of polymers was also confirmed with XRM, showing a cross-section of the microparticles (right).

The degradation of the microparticles was also studied. Specifically, the in vitro biodegradation of LBL MPs at different pHs was observed (FIG. 5B). At pH 5.5, there were visible surface erosion post week 5. By week 13, surface erosion has begun to release the PLLA/PLGA pellets incorporated in the shell. By week 24, the majority of the particles have either disintegrated completely or have progressed with the surface erosion to the point where integrated pellets have been released from the shell.

At pH 7.5, surface erosion began earlier, as shown by the visible protrusion of the PLLA/PLGA pellets. However, similar to the lower pH, surface erosion was extensive to the point that many particles became fragile and have begun to break. By week 24, there were no particles to be seen, marking the completion of the particle breakdown.

Overall, particle degradation is accelerated at pH 7.5. This is owing to the fact that the polymers are acidic components and with the increased contact surface with the biofluids due to the pores, disintegration of the particles is rapid and complete by week 24.

Based on the foregoing, the actual degradation of the particles may be at an accelerated pace in vivo because (i) continuous exchange of the conditions and (ii) presence of bio-secretory fluids, including enzymes, that may catalyze the polymer degradation to occur at a much faster rate.

Lastly, rats were injected intramuscularly with LBL-MPs (loaded with antigen). Plasma samples obtained from the rats were subjected to enzyme linked immunosorbent assay (ELISA) to quantify the IgG against SARS-CoV-2 nucleoprotein at days 7 and 28. As seen in FIG. 5C, there were significant quantities of anti-SArS-CoV-2 IgG for nucleoprotein found in the rat plasma, indicative of immune stimulation.

### Example 2

Microparticles with different porogens were synthesized by the following protocol.
1. PCL (200 mg) was dissolved in 2 ml DCM. 200 μL of 2-methylpentane or 3-methylpentane or 2-methylhexane or 3-methylhexane or 2,4-dimethylpentane or 2,3-dimethylbutane was mixed thoroughly into the DCM-polymer mix.
2. The resulting polymer mixture was poured dropwise into 1% w/v polyvinyl alcohol (300 ml) aqueous solution while being stirred at 1000 rpm.
3. The mechanical stirring (at max speed 1300 rpm) at 25.0° C. was continued for 6-16 hours to completely evaporate the organic solvent and to obtain the solid microparticles.
4. The solid microparticles were centrifuged at 2000 rpm (alternatively filtered using Whatman paper). The pellet was lyophilized and subjected to characterization.
5. The same procedure was repeated for two other polymers: poly lactic-co-glycolic acid (PLGA) and poly-L-lactic acid (PLLA), respectively.
6. Porous microparticle morphology was directly observed with scanning electron microscopy (SEM). The SEM images were analyzed using ImageJ for particle size and pore size distribution.

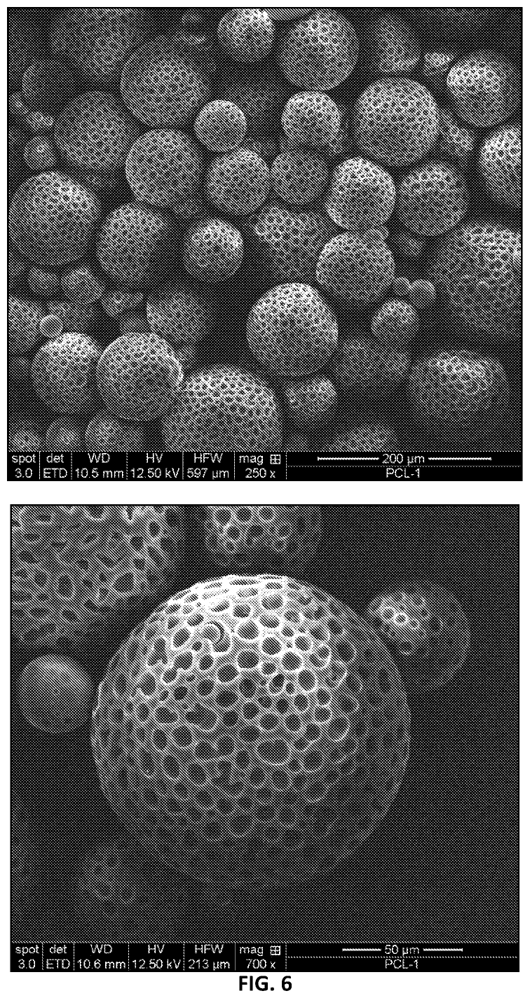

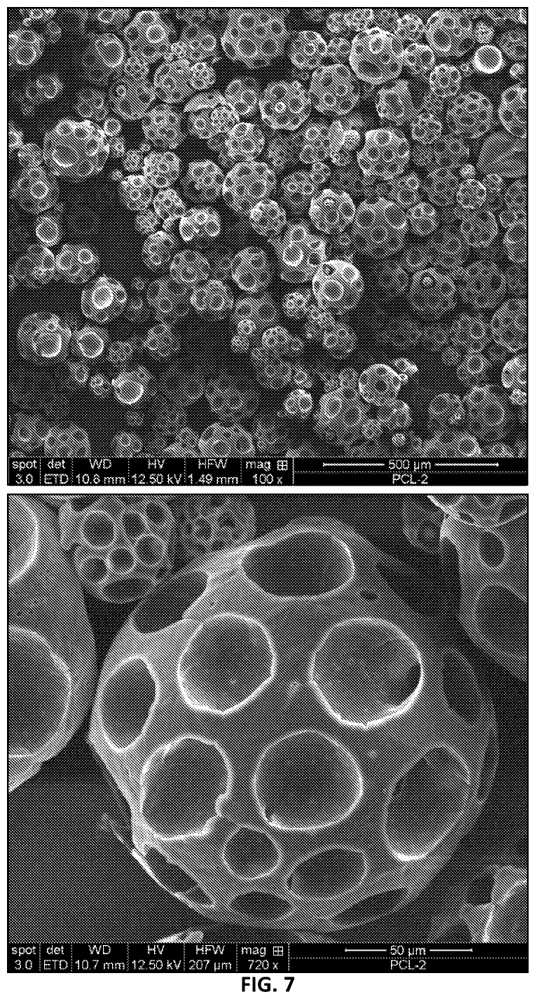

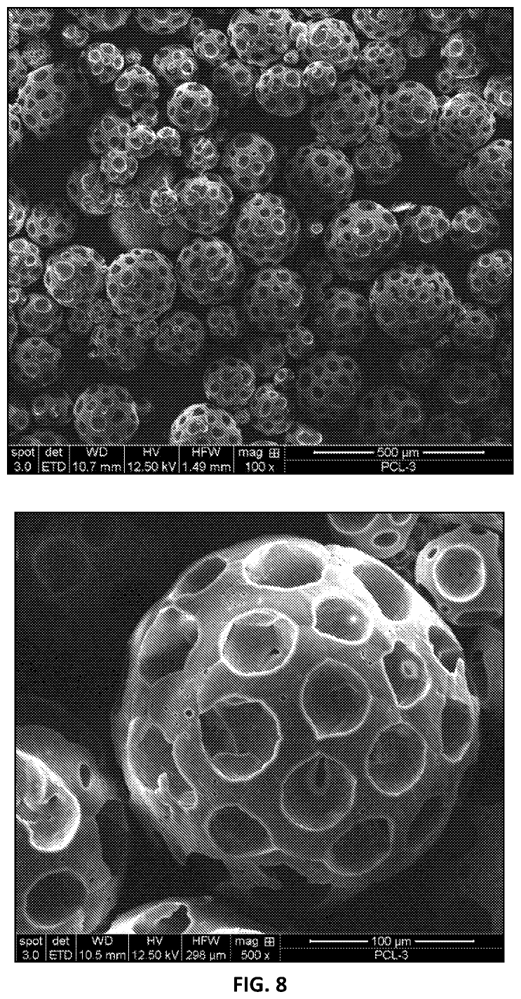

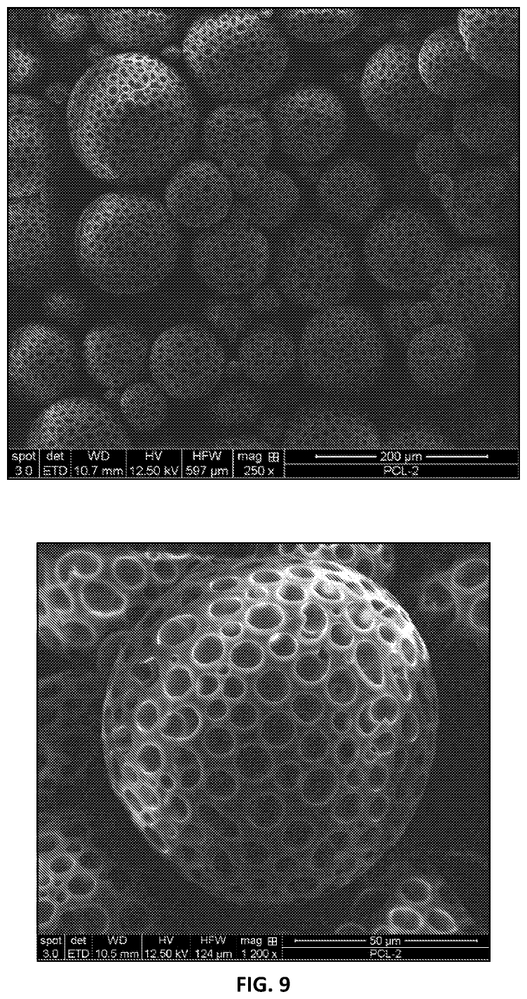

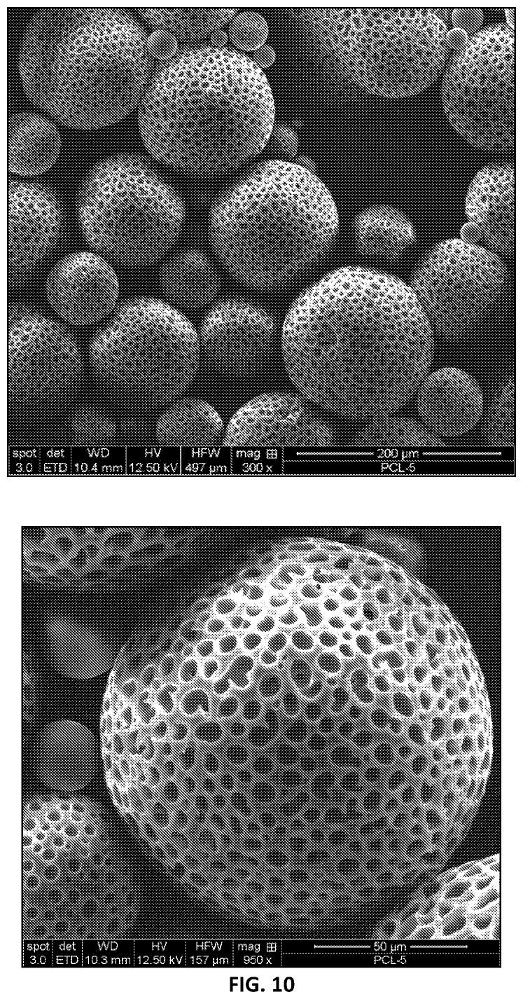

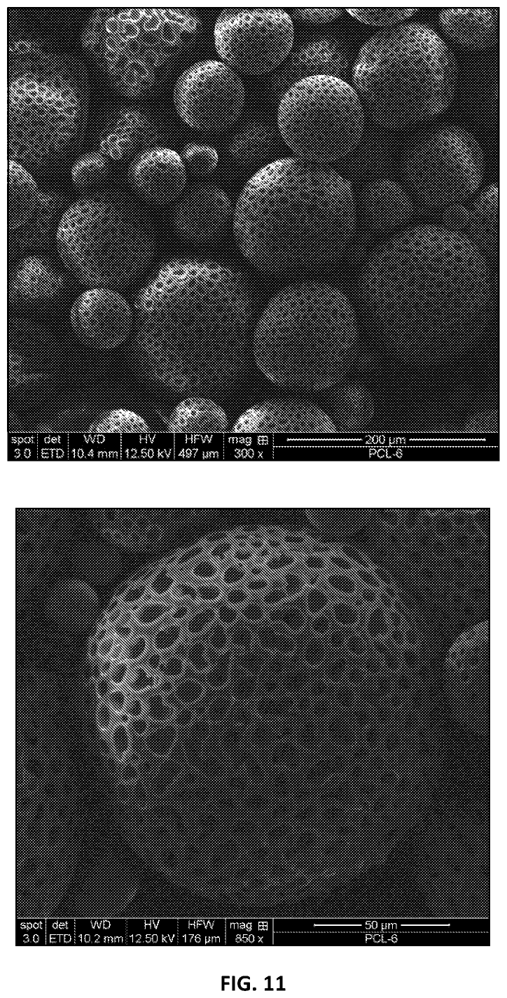

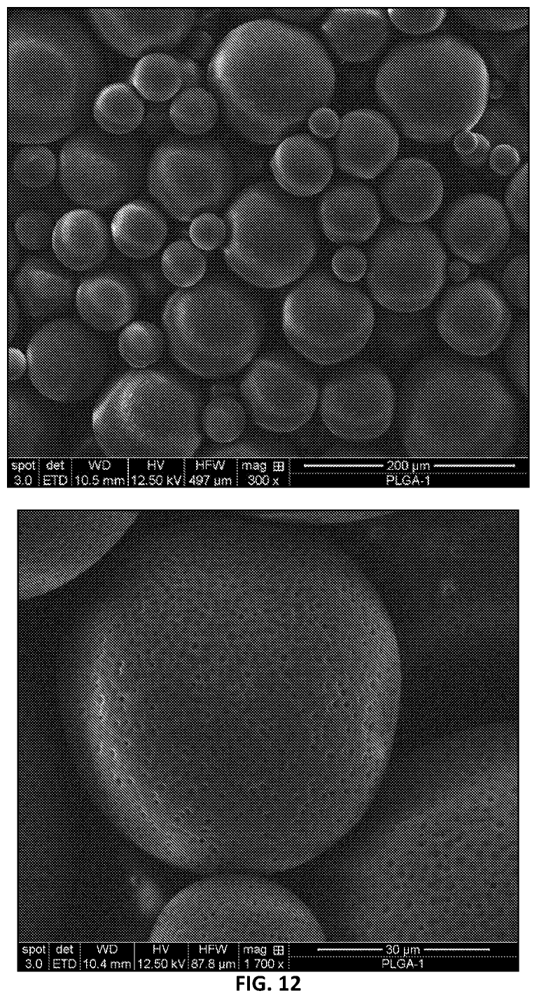

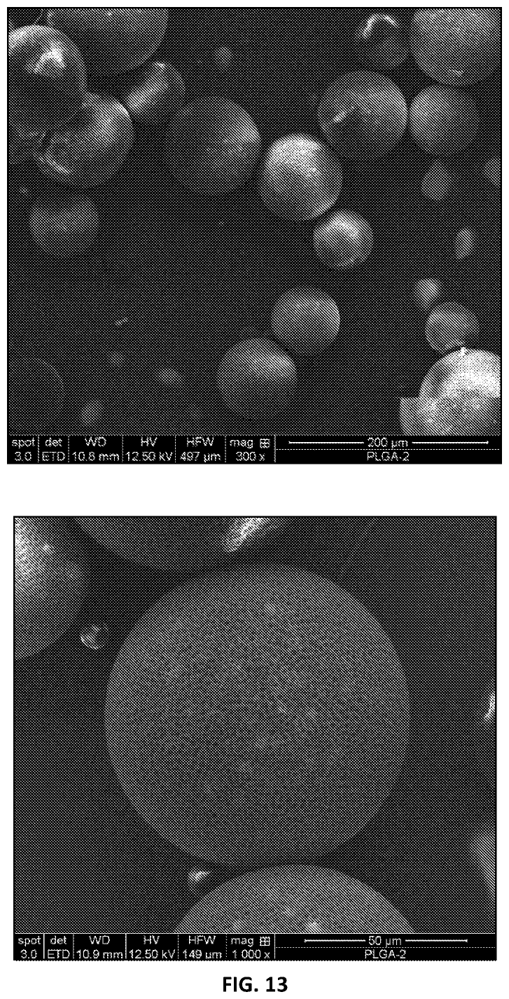

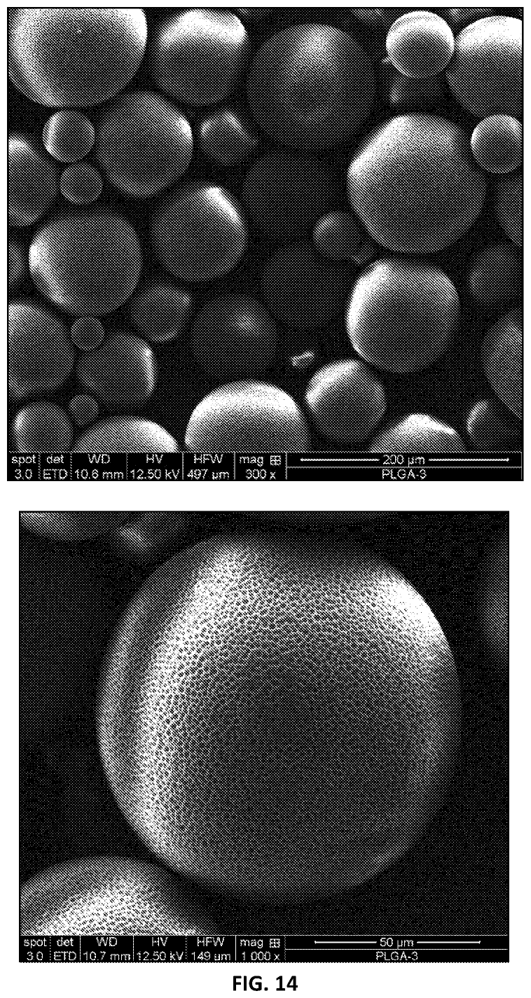

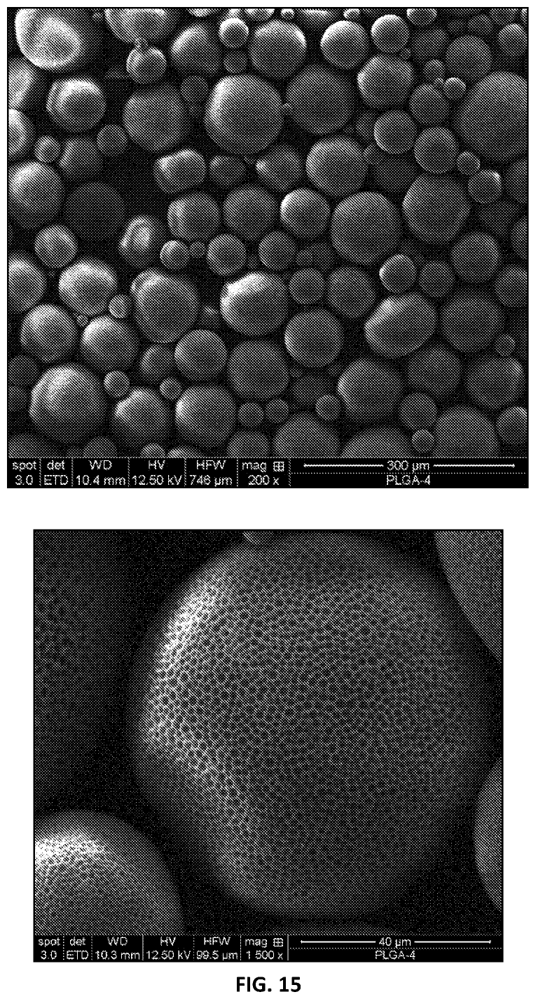

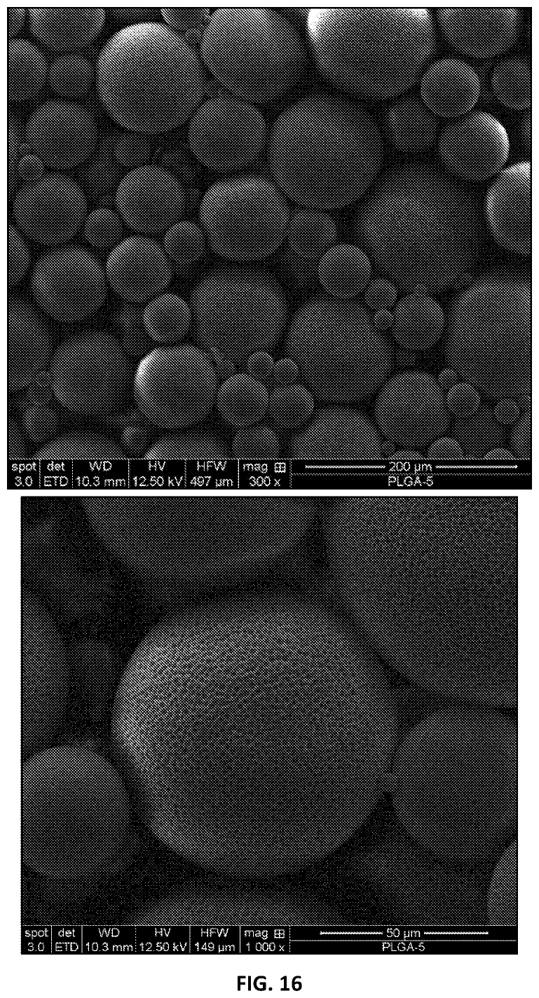

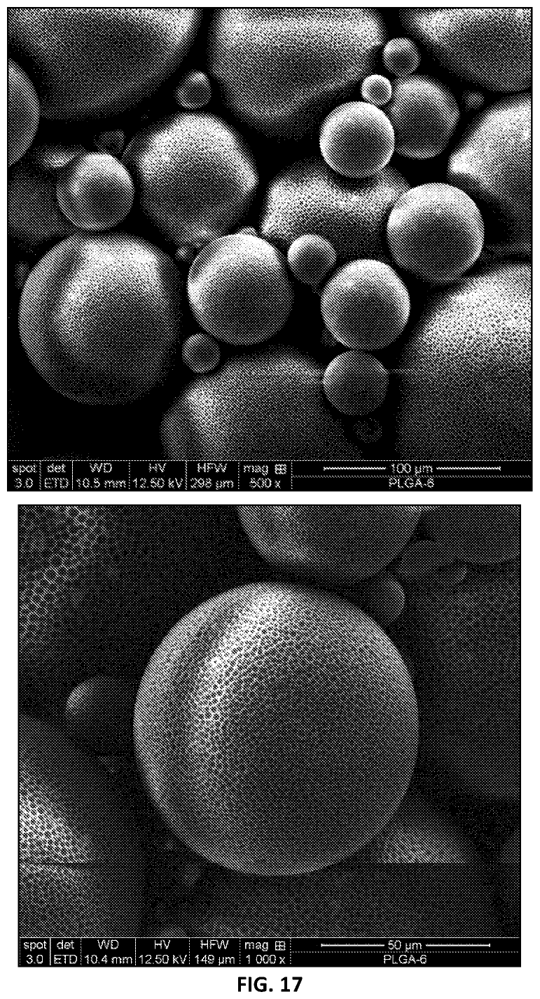

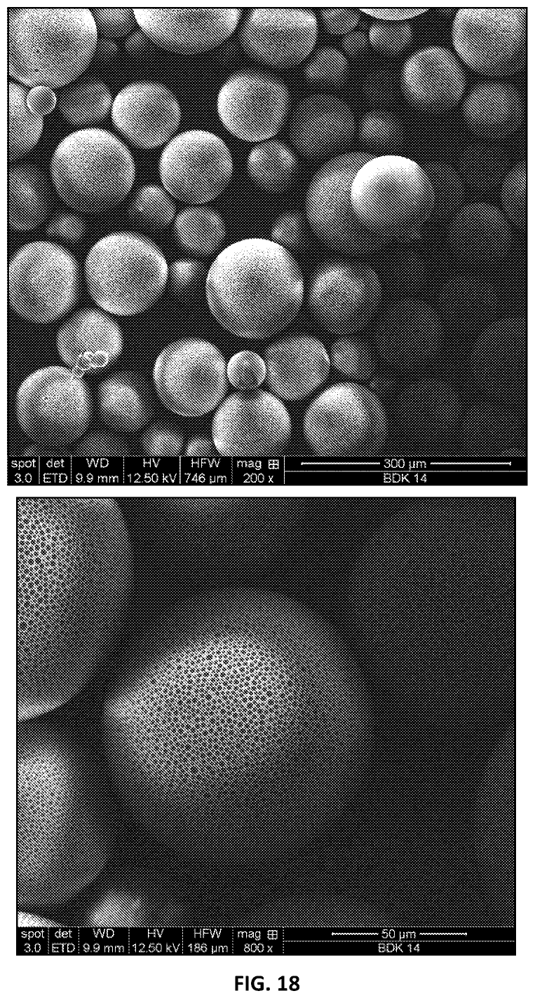

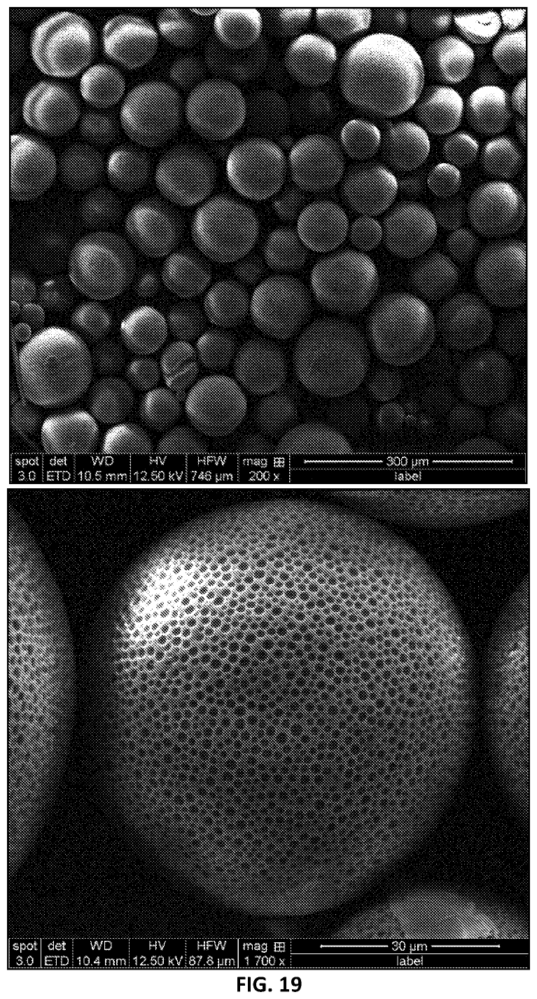

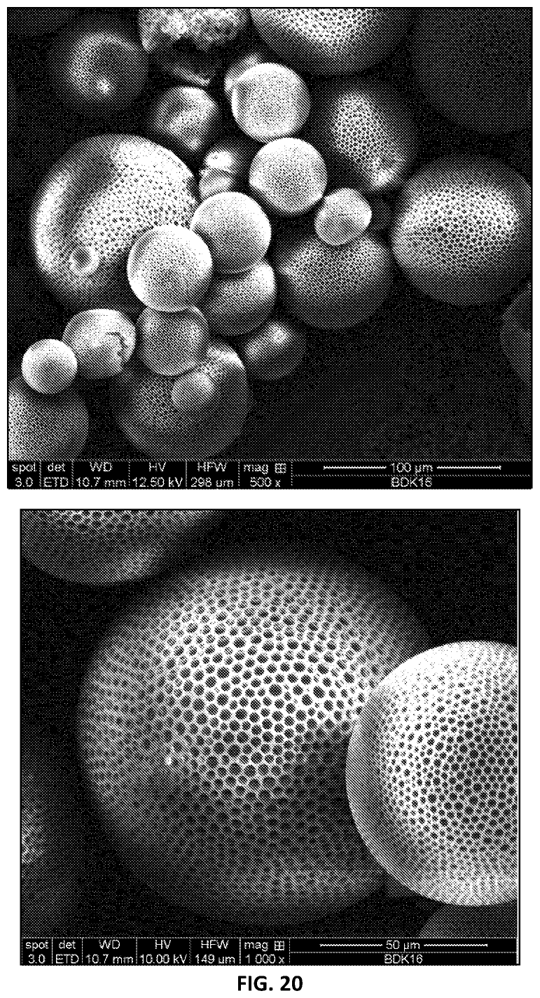

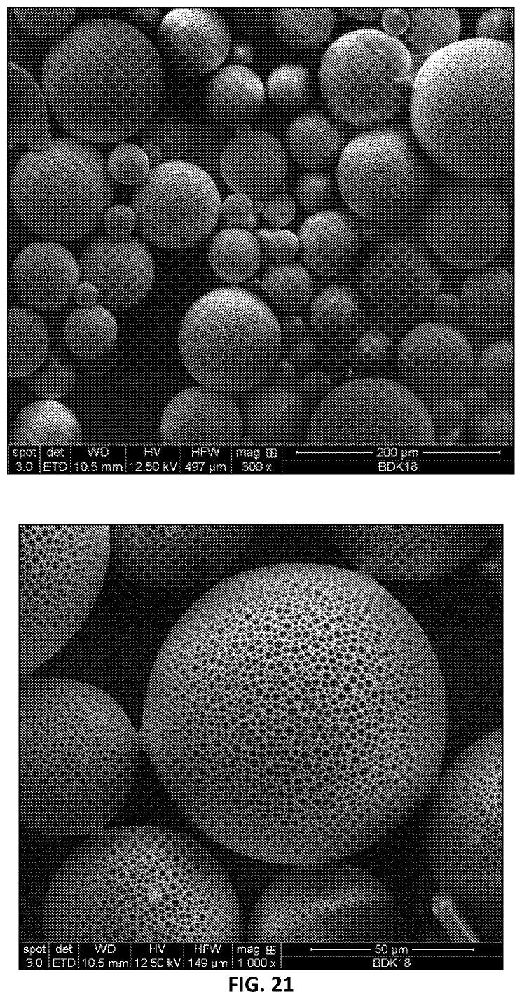

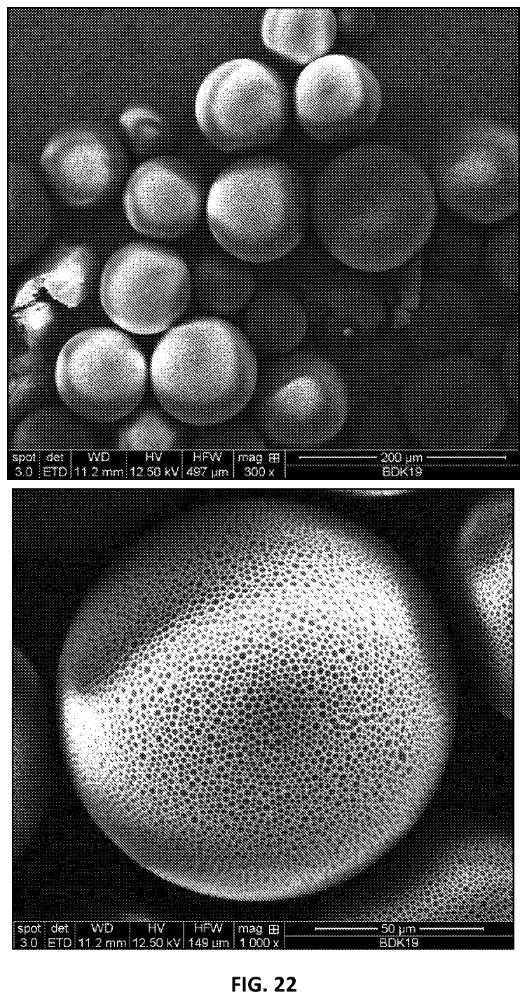

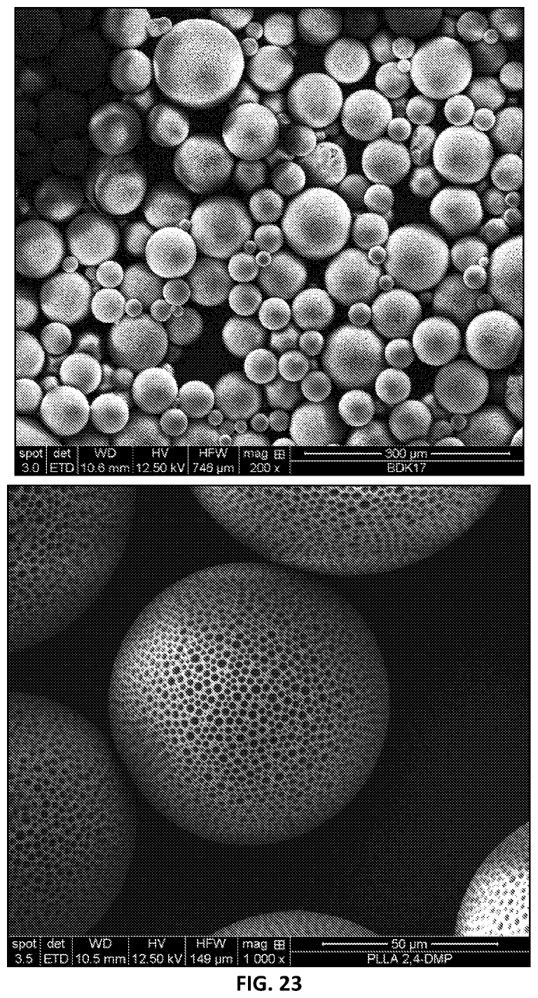

Example polymer and porogen combinations to produce
particles with specific pore sizes.
A microparticle composed of the polymer PCL produced with the
porogen 2-MP will create pore sizes between 1277 nm and 2007 nm.
(FIG. 6)
A microparticle composed of the polymer PCL produced with the
porogen 3-MP will create pore sizes between 854 nm and 1709 nm.
(FIG. 7)
A microparticle composed of the polymer PCL produced with the
porogen 2-MH will create pore sizes between 1614 nm and 3655 nm.
(FIG. 8)
A microparticle composed of the polymer PCL produced with the
porogen 3-MH will create pore sizes between 1312 nm and 2478 nm.
(FIG. 9)
A microparticle composed of the polymer PCL produced with the
porogen 2,3-DMB will create pore sizes between 1020 nm and 2915 nm.
(FIG. 10)
A microparticle composed of the polymer PCL produced with the
porogen 2,4-DMP will create pore sizes between 1020 nm and 3352 nm.
(FIG. 11)
A microparticle composed of the polymer PLGA produced with the
porogen 2-MP will create pore sizes between 429 nm and 1375 nm.
(FIG. 12)
A microparticle composed of the polymer PLGA produced with the
porogen 3-MP will create pore sizes between 1220 nm and 2848 nm.
(FIG. 13)
A microparticle composed of the polymer PLGA produced with the
porogen 2-MH will create pore sizes between 935 nm and 1900 nm.
(FIG. 14)
A microparticle composed of the of polymer PLGA produced with the
porogen 3-MH will create pore sizes between 994 nm and 1812 nm.
(FIG. 15)
A microparticle composed of the polymer PLGA produced with the
porogen 2,3-DMB will create pore sizes between 494 nm and 1976 nm.
(FIG. 16)
A microparticle composed of the polymer PLGA produced with the
porogen 2,4-DMP will create pore sizes between 1075 nm and 2151 nm.
(FIG. 17)
A microparticle composed of the polymer PLLA produced with the
porogen 2-MP will create pore sizes between 1094 nm and 2007 nm.
(FIG. 18)
A microparticle composed of the polymer PLLA produced with the
porogen 3-MP will create pore sizes between 854 nm and 1709 nm.
(FIG. 19)
A microparticle composed of the polymer PLLA produced with the
porogen 2-MH will create pore sizes between 1614 nm and 3655 nm.
(FIG. 20)
A microparticle composed of the polymer PLLA produced with the
porogen 3-MH will create pore sizes between 1312 nm and 2478 nm.
(FIG. 21)
A microparticle composed of the polymer PLLA produced with the
porogen 2,3-DMB will create pore sizes between 1020 nm and 2915 nm.
(FIG. 22)
A microparticle composed of the polymer PLLA produced with the
porogen 2,4-DMP will create pore sizes between 1020 nm and 3352 nm.
(FIG. 23)

A number of publications and patent documents are cited throughout the foregoing specification in order to describe the state of the art to which this invention pertains. The entire disclosure of each of these citations is incorporated by reference herein.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1. A microparticle comprising one or more polymer layers, wherein said microparticle comprises pores and at least one therapeutic agent.

2. The microparticle of claim 1, wherein said microparticle comprises more than one polymer layer.

3. The microparticle of claim 1, wherein said polymer layer comprises hydrophobic polymers.

4. The microparticle of claim 1, wherein said polymer layer comprises poly(lactide-co-glycolide) (PLGA), poly(L-lactide) (PLLA), or polycaprolactone (PCL).

5. The microparticle of claim 1, wherein the microparticle comprises an external layer of polycaprolactone (PCL) and an internal core of poly(lactide-co-glycolide) (PLGA).

6. The microparticle of claim 1, wherein each polymer layer comprises a therapeutic agent.

7. The microparticle of claim 1, wherein the therapeutic agent is a vaccine.

8. The microparticle of claim 7, wherein the vaccine is selected from the group consisting of inactivated virus, attenuated virus, viral protein, viral peptide, viral polysaccharide, viral capsid, viral RNA, viral DNA, viral vectors, and combinations thereof.

9. The microparticle of claim 7, wherein the vaccine is an inactivated virus.

10. The microparticle of claim 9, wherein the inactivated virus is inactivated SARS-CoV-2.

11. A composition comprising at least microparticle of claim 1 and a pharmaceutically acceptable carrier.

12. A method of treating, inhibiting, and/or preventing a disease in a subject in need thereof, said method comprising administering to said subject a microparticle of claim 1.

13. The method of claim 12, wherein said disease is an infectious disease.

14. The method of claim 13, wherein said infection disease is a viral infection.

15. A method of synthesizing a microparticle, said method comprising:
a) dissolving at least one polymer and at least one porogen in an organic solvent,
b) adding the polymer and porogen mixture to an aqueous solution under stirring until the organic solution evaporates, and
c) isolating the resultant microparticles.

16. The method of claim 15, wherein said aqueous solution comprises polyvinyl alcohol.

17. The method of claim 15, further comprising adding an agent to be incorporated into the microparticle.

18. The method of claim 17, wherein said agent is added to the aqueous solution.

19. The method of claim 17, wherein said agent is an inactivated virus.

20. The method of claim 19, further comprising the step of inactivating the virus.

21. The method of claim 15, wherein said porogen is a branched chain alkane or hydrocarbon comprising 3-10 carbons.

22. The method of claim 15, wherein said porogen is selected from the group consisting of isohexane, 2,4-dimethylpentane, 2,5-dimethylhexane, 2-methylheptane, 2-methylhexane, 2,4-dimethylhexane, 2-methypentane (2-MP), 3-methylpentane (3-MP), 2-methylhexane (2-MH), 3-methylhexane (3-MH), 2,4-dimethylpentane (2,4-DMP), 2,3-dimethylbutane (2,3-DMB), 2-butanone, and heptane.

23. The method of claim 15, wherein said porogen is selected from the group consisting of 2-methypentane (2-MP), 3-methylpentane (3-MP), 2-methylhexane (2-MH), 3-methylhexane (3-MH), 2,4-dimethylpentane (2,4-DMP), and 2,3-dimethylbutane (2,3-DMB).

24. A method for controlling or modulating the pore size and/or frequency or pores of a microparticle, said method comprising changing the porogen, polymer, and/or solvent used in the synthesis of the microparticle and/or modulating the amount of porogen used in the synthesis of the microparticle.
